(19) United States
(12) Patent Application Publication
(10) Pub. No.: US 2010/0029706 A1
(43) Pub. Date: Feb. 4, 2010

(54) HYDROGENATED PYRIDO[4,3-b]INDOLES FOR THE TREATMENT OF OXIDATIVE STRESS

(75) Inventors: Guy M. Miller, Monte Sereno, CA (US); Kieron E. Wesson, Burlingame, CA (US)

Correspondence Address: WILSON, SONSINI, GOODRICH & ROSATI, 650 PAGE MILL ROAD, PALO ALTO, CA, 94304-1050, US

(73) Assignee: Edison Parmaceuticals, Inc. a Delaware Corporation, San Jose, CA (US)

(21) Appl. No.: 12/511,934

(22) Filed: Jul. 29, 2009

Related U.S. Application Data
(60) Provisional application No. 61/137,339, filed on Jul. 30, 2008.

(51) Int. Cl. A61K 31/437; A61P 43/00

(52) U.S. Cl. 514/292

(57) ABSTRACT

Methods of treating or suppressing oxidative stress diseases including mitochondrial diseases, impaired energy processing disorders, and diseases of aging such as diabetes and cancer with hydrogenated pyrido[4,3-b]indoles such as dimebolin, are disclosed.

### CROSS-REFERENCE

This application claims priority benefit of U.S. Provisional Patent Application No. 61/137,339, filed Jul. 30, 2008. The entire content of that application is hereby incorporated by reference herein in its entirety.

### BACKGROUND OF THE INVENTION

Oxidative stress is caused by disturbances to the normal redox state within cells. An imbalance between routine production and detoxification of reactive oxygen species such as peroxides and free radicals can result in oxidative damage to cellular structures and machinery. Under normal conditions, an important source of reactive oxygen species in aerobic organisms is likely the leakage of activated oxygen from mitochondria during normal oxidative respiration. Impairments associated with this process may contribute to mitochondrial disease. Therapeutics that target oxidative stress could potentially benefit patients suffering from a variety of diseases.

### SUMMARY OF THE INVENTION

In a first aspect of the invention, a method is provided to treat a subject having an oxidative stress disorder, or at risk for having an oxidative stress disorder, comprising administering to the subject a therapeutically effective amount of one or more compounds of Formula I:

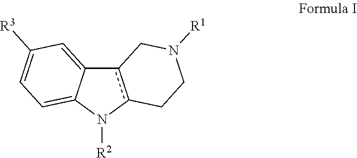

or its pharmaceutically acceptable salts, prodrugs, solvates, or hydrates thereof; wherein R¹ is hydrogen, alkyl, aralkyl or heteroaralkyl; R² is hydrogen, aralkyl, or heteroaralkyl; R³ is hydrogen, alkyl, or halo; a bond represented by a solid line accompanied by a dotted line is a single or a double bond; wherein the oxidative stress disorder is a haemoglobinopathy or caused by a defect in a gene encoding a mitochondrial protein or tRNA; and wherein the oxidative stress disorder is not Leber's Hereditary Optic Neuropathy (LHON).

In a second aspect of the invention, a method is provided to modulate the level of energy biomarkers in a subject comprising administering to the subject an effective amount of one or more compounds wherein the one or more compounds normalizes one or more energy markers in a subject or enhances or reduces the level of each of one or more energy biomarkers in the subject by more than 10%.

In some embodiments of the second aspect, the one or more compounds administered to a subject have the structure of Formula I:

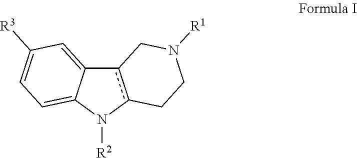

or its pharmaceutically acceptable salts, prodrugs, solvates, or hydrates thereof; wherein R¹ is hydrogen, alkyl, aralkyl or heteroaralkyl; R² is hydrogen, aralkyl, or heteroaralkyl; R³ is hydrogen, alkyl, or halo; and the bond represented by a solid line accompanied by a dotted line is a single or a double bond.

In various embodiments of any of the aspects of the invention, the method comprises measuring the level of one or more energy biomarkers in the subject prior to or following the administering of the compound. In some embodiments, the one or more energy biomarkers is selected from the group consisting of: lactic acid (lactate) levels; pyruvic acid (pyruvate) levels; lactate/pyruvate ratios; phosphocreatine levels NADH (NADH+H⁺) levels; NADPH (NADPH+H⁺) levels; NAD levels; NADP levels; ATP levels; reduced coenzyme Q (CoQ^{red}) levels; oxidized coenzyme Q (CoQ^{ox}) levels; total coenzyme Q (CoQ^{tot}) levels; oxidized cytochrome C levels; reduced cytochrome C levels; oxidized cytochrome C/reduced cytochrome C ratio; acetoacetate levels; β-hydroxy butyrate levels; acetoacetate/β-hydroxy butyrate ratio, 8-hydroxy-2′-deoxyguanosine (8-OHdG) levels; levels of reactive oxygen species; levels of oxygen consumption (VO₂) and levels of carbon dioxide output (VCO₂). In some embodiments, the subject has an abnormal level of one or more energy biomarkers. In yet other embodiments, the subject has a normal level of one or more energy biomarkers. In further embodiments, the subject has an abnormal respiratory quotient (VCO₂/VO₂), an abnormal result from an exercise tolerance test, or an abnormal anaerobic threshold.

In a third aspect of the invention, a method is provided to treat a subject having an oxidative stress disorder comprising: (a) testing the subject for a genetic defect; and (b) administering to the subject a therapeutically effective amount of one or more compounds of Formula I:

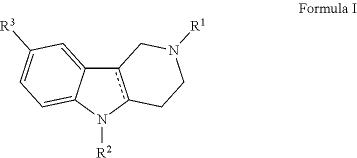

or its pharmaceutically acceptable salts, prodrugs, solvates, or hydrates thereof; wherein R¹ is hydrogen, alkyl, aralkyl or heteroaralkyl; R² is hydrogen, aralkyl, or heteroaralkyl; R³ is hydrogen, alkyl, or halo; and a bond represented by a solid line accompanied by a dotted line is a single or a double bond.

In a fourth aspect of the invention, a formulation is provided comprising a first and second compound wherein the first compound is effective against an oxidative stress disorder and the second compound is one or more compounds of Formula I:

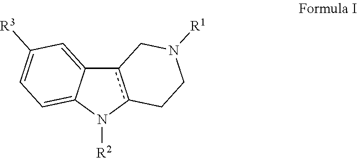

or its pharmaceutically acceptable salts, prodrugs, solvates, or hydrates thereof; wherein R¹ is hydrogen, alkyl, aralkyl or heteroaralkyl; R² is hydrogen, aralkyl, or heteroaralkyl; R³ is hydrogen, alkyl, or halo; and a bond represented by a solid line accompanied by a dotted line is a single or a double bond.

In some embodiments of the fourth aspect, the first compound is effective against haemoglobinopathy or a disease or disorder caused by a defect in a gene encoding a mitochondrial protein or tRNA. In other embodiments, the first compound is selected from the group consisting of: vitamin, antioxidant compound, iron chelator, antioxidant used to reduce preferryl-Hb, indicaxanthin, a drug used to lower lung hypertension, Gardos channel blocker, a drug used to modify hemoglobin switching, a drug used to treat vaso-occlusive crises, analgesic, NSAID, opiod, and antibiotic. In yet other embodiments, the first compound is selected from the group consisting of: erthyropoietin, erythropoietin mutant, erythropoietin biosimilar, erythropoietin mimetic, Coenzyme Q, vitamin E, Idebenone, MitoQ, deferoxamine, deferasirox, indicaxanthin, sildenafil, nifedine, hydroxyurea, seniapoc, phytochemical, nicosan; folic acid, quinolone and macrolide. In some embodiments, the formulation further comprises a pharmaceutically acceptable excipient.

In some embodiments of any of the aspects of the invention, an oxidative stress disorder is caused by a defect in a gene encoding a mitochondrial protein or tRNA. In other embodiments, the defect in a gene encoding a mitochondrial protein or tRNA results in a respiratory chain disorder. In yet other embodiments, the defect in a gene encoding a mitochondrial protein or tRNA causes a disorder selected from the group consisting of Myoclonic Epilepsy with Ragged Red Fibers (MERRF); Mitochondrial Myopathy, Encephalopathy, Lactacidosis, Stroke (MELAS); chronic progressive external ophthalmoplegia (CPEO); Leigh Disease; Kearns-Sayre Syndrome (KSS); Friedreich's Ataxia (FRDA); Co-Enzyme Q10 (CoQ10) Deficiency; Complex I Deficiency; Complex II Deficiency; Complex III Deficiency; Complex IV Deficiency; and Complex V Deficiency. Alternatively, the invention provides embodiments of the first aspect wherein the oxidative stress disorder is a haemoglobinopathy. In some embodiments, wherein the oxidative stress disorder is a haemoglobinopathy, the haemoglobinopathy is thalassemia or sickle-cell disease.

In various embodiments of the first aspect, the oxidative stress disorder is not a neurodegenerative disease. In some other embodiments, the oxidative stress disorder is not Huntington's disease, amyotrophic lateral sclerosis (ALS), Parkinson's disease, Alzheimer's Disease, or neuronal death mediated ocular disease. In further embodiments, the oxidative stress disorder is not Leber's Hereditary Optic Neuropathy (LHON). In some embodiments, the oxidative stress disorder is not Huntington's disease. In other embodiments, the oxidative stress disorder is not ALS. In yet other embodiments, the oxidative stress disorder is not Parkinson's Disease. The invention provides other embodiments wherein the oxidative stress disorder is not a neuronal-mediated ocular disease. In various embodiments, the oxidative stress disorder is not Alzheimer's Disease.

In some of the embodiments of the invention, the one or more compounds of Formula I are administered with a pharmaceutically acceptable excipient.

In any of the aspects of the invention, the compound of Formula I is a compound having a structure of Formula I-A or Formula I-B:

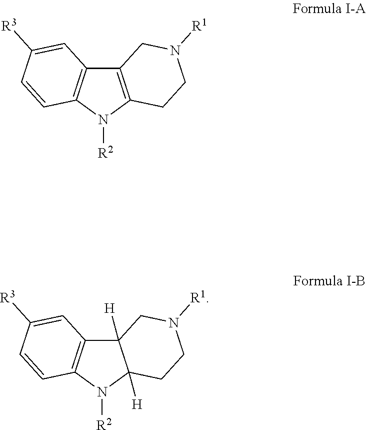

In some embodiments of the compound of Formula I, when R¹ and/or R³ is alkyl, the R¹ and/or R³ is methyl. In other embodiments, when R¹ and/or R² is an aralkyl moiety, the aryl of the aralkyl moiety is phenyl and the alkyl of the aralkyl moiety is methyl. In further embodiments, when R¹ and/or R² is a heteroaralkyl moiety, the heteroaryl of the heteroaralkyl moiety is pyridinyl. Alternatively, the invention provides for compounds of Formula I wherein R¹ is hydrogen, C₁-C₄-alkyl, benzyl or 3-(pyridin-3-yl)propyl; R² is hydrogen, benzyl or 2-(6-methylpyridin-3-yl)ethyl); R³ is hydrogen, C₁-C₄-alkyl, or halogen. In some embodiments, R¹ is C₁-C₄-alkyl, benzyl or 3-(pyridin-3-yl)propyl. In some other embodiments, when R¹ and/or R³ is C₁-C₄-alkyl, the C₁-C₄-alkyl is unsubstituted.

In other embodiments of the compound of Formula I, the compound is selected from the group consisting of dimebolin (2,8-dimethyl-5-(2-(6-methylpyridin-3-yl)ethyl)-2,3,4,5-tetrahydro-1H-pyrido[4,3-b]indole)(also known as “Dimebon”); 8-chloro-2-methyl-5-(2-(6-methylpyridin-3-yl)ethyl)-2,3,4,5-tetrahydro-1H-pyrido[4,3-b]indole; mebhydroline (5-benzyl-2-methyl-2,3,4,5-tetrahydro-1H-pyrido[4,3-b]indole); 2,8-dimethyl-1,3,4,4a,5,9b-hexahydro-1H-pyrido[4,3-b]indole; 8-fluoro-2-(3-(pyridin-3-yl)propyl)-2,3,4,5-tetrahydro-1H-pyrido[4,3-b]indole; and 8-methyl-1,3,4,4a,5,9b-tetrahydro-1H-pyrido[4,3-b]indole. In some embodiments, when the compound of Formula I is 2,8-dimethyl-1,3,4,4a,5,9b-hexahydro-1H-pyrido[4,3-b]indole, the compound is a mixture of (+/−)cis isomers (Carbidine); a (−)cis isomer (Stobadine, having stereochemistry (4aR, 9bS) as shown in Formula I-B4); any one of (4aS, 9bR), (4aS, 9bS), (4aR, 9bR), and (4aR, 9bS) isomers; or a mixture of (4aS, 9bR), (4aS, 9bS), (4aR, 9bR), and/or (4aR, 9bS) isomers in any proportion thereof In other embodiments, the compound is dimebolin (2,8-dimethyl-5-(2-(6-methylpyridin-3-yl)ethyl)-2,3,4,5-tetrahydro-1H-pyrido[4,3-b]indole). In further embodiments, the compound is a hydrochloride, sulfate, phosphate, fumarate, maleate, palmitate, tosylate, mesylate, acetate, or citrate salt.

In a fifth aspect of the invention, a method is provided to treat a subject having an oxidative stress disorder comprising administering to the subject a therapeutically effective amount of one or more compounds of Formula I:

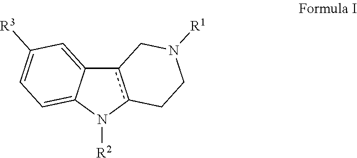

or its pharmaceutically acceptable salts, prodrugs, solvates, or hydrates thereof; wherein R¹ is hydrogen, alkyl, aralkyl or heteroaralkyl; R² is hydrogen, aralkyl, or heteroaralkyl; R³ is hydrogen, alkyl, or halo; a bond represented by a solid line accompanied by a dotted line is a single or a double bond; wherein the oxidative stress disorder is a haemoglobinopathy or caused by a defect in a gene encoding a mitochondrial protein or tRNA; and wherein the oxidative stress disorder is not Leber's Hereditary Optic Neuropathy (LHON).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

The instant application discloses compositions of hydrogenated pyrido[4,3-b]indole derivatives (e.g., dimebolin), and methods useful for treatment, prevention, or suppression of diseases, disorders, developmental delays and symptoms related to oxidative stress affecting normal electron flow in cells. Nonlimiting examples of such diseases are mitochondrial disorders, haemoglobinopathies, impaired energy processing disorders, and diseases of aging such as diabetes and cancer. Notably, provided herein are tetra- or hexahydro-1H-pyrido[4,3-b]indole derivatives that can treat diseases and/or disorders related to oxidative stress affecting normal electron flow in the cells, such as mitochondrial diseases and impaired energy processing disorders.

The ability to adjust biological production of energy has applications beyond specific diseases. Various other disorders can result in suboptimal levels of energy biomarkers (sometimes also referred to as indicators of energetic function), such as ATP levels. Treatments for these disorders are also needed, in order to modulate one or more energy biomarkers to improve the health of the patient. In other applications, it can be desirable to modulate certain energy biomarkers away from their normal values in an individual that is not suffering from disease. For example, if an individual is undergoing an extremely strenuous undertaking, it can be desirable to raise the level of ATP in that individual.

By “subject,” “individual,” or “patient” is meant an individual organism, preferably a vertebrate, more preferably a mammal, most preferably a human.

The term “effective amount” or “therapeutically effective amount” refers to that amount of a compound described herein that is sufficient to effect the intended application including but not limited to treatment of a disease, disorder or condition. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g. reduction in oxidative stress or modulation, normalization, or enhancement of one or more energy biomarkers in a subject. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating,” or “palliating” or “ameliorating” are used interchangeably herein. These terms refers to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the patient may still be afflicted with the underlying disorder. For prophylactic benefit, the compositions may be administered to a subject at risk of developing a particular disease, or to a subject reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

By “respiratory chain disorder” is meant a disorder which results in the decreased utilization of oxygen by a mitochondrion, cell, tissue, or individual, due to a defect or disorder in a protein contained in the mitochondrial respiratory chain. By “respiratory chain” is meant the components (including, but not limited to, proteins, tetrapyrroles, and cytochromes) comprising mitochondrial complex I, II, III, IV, and/or V; “respiratory chain protein” refers to the protein components of those complexes.

“Modulation” of, or to “modulate,” an energy biomarker means to change the level of the energy biomarker towards a desired value, or to change the level of the energy biomarker in a desired direction (e.g., increase or decrease). Modulation can include, but is not limited to, normalization and enhancement as defined below.

“Normalization” of, or to “normalize,” an energy biomarker is defined as changing the level of the energy biomarker from a pathological value towards a normal value, where the normal value of the energy biomarker can be 1) the level of the energy biomarker in a healthy person or subject, or 2) a level of the energy biomarker that alleviates one or more undesirable symptoms in the person or subject. That is, to normalize an energy biomarker which is depressed in a disease state means to increase the level of the energy biomarker towards the normal (healthy) value or towards a value which alleviates an undesirable symptom; to normalize an energy biomarker which is elevated in a disease state means to decrease the level of the energy biomarker towards the normal (healthy) value or towards a value which alleviates an undesirable symptom.

“Enhancement” of, or to “enhance,” energy biomarkers means to intentionally change the level of one or more energy biomarkers away from either the normal value, or the value before enhancement, in order to achieve a beneficial or desired effect. For example, in a situation where significant energy demands are placed on a subject, it may be desirable to increase the level of ATP in that subject to a level above the normal level of ATP in that subject. Enhancement can also be of beneficial effect in a subject suffering from a disease or pathology such as a mitochondrial disease, in that normalizing an energy biomarker may not achieve the optimum outcome for the subject; in such cases, enhancement of one or more energy biomarkers can be beneficial, for example, higher-than-normal levels of ATP, or lower-than-normal levels of lactic acid (lactate) can be beneficial to such a subject.

By modulating, normalizing, or enhancing the energy biomarker Coenzyme Q is meant modulating, normalizing, or enhancing the variant or variants of Coenzyme Q which is predominant in the species of interest. For example, the variant of Coenzyme Q which predominates in humans is Coenzyme Q10. If a species or subject has more than one variant of Coenzyme Q present in significant amounts (i.e., present in amounts which, when modulated, normalized, or enhanced, can have a beneficial effect on the species or subject), modulating, normalizing, or enhancing Coenzyme Q can refer to modulating, normalizing or enhancing any or all variants of Coenzyme Q present in the species or subject.

The term “Friedreich's ataxia” is also sometimes referred to as hereditary ataxia, familiar ataxia, or Friedreich's tabes.

The term “Ataxia” is an aspecific clinical manifestation implying dysfunction of parts of the nervous system that coordinate movement, such as the cerebellum. People with ataxia have problems with coordination because parts of the nervous system that control movement and balance are affected. Ataxia may affect the fingers, hands, arms, legs, body, speech, and eye movements. The word ataxia is often used to describe a symptom of incoordination which can be associated with infections, injuries, other diseases, or degenerative changes in the central nervous system. Ataxia is also used to denote a group of specific degenerative diseases of the nervous system called the hereditary and sporadic ataxias. Ataxias are also often associated with hearing impairments.

The term “in vivo” refers to an event that takes place in a subject's body.

The term “in vitro” refers to an event that takes places outside of a subject's body. For example, an in vitro assay encompasses any assay run outside of a subject assay. In vitro assays encompass cell-based assays in which cells alive or dead are employed. In vitro assays also encompass a cell-free assay in which no intact cells are employed.

The term “co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions of the invention is contemplated. Supplementary active ingredients can also be incorporated into the compositions.

The compounds described herein can occur and can be used as the neutral (non-salt) compound. Alternatively, the description is intended to embrace all salts of the compounds described herein, as well as methods of using such salts of the compounds. In one embodiment, the salts of the compounds comprise pharmaceutically acceptable salts. Pharmaceutically acceptable salts are those salts which can be administered as drugs or pharmaceuticals to humans and/or animals and which, upon administration, retain at least some of the biological activity of the free compound (neutral compound or non-salt compound). The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, can also be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include, but are not limited to, procaine, dibenzylamine, N-ethylpiperidine, N,N-dibenzylethylenediamine, and triethylamine salts. Salts of acidic compounds with amino acids, such as lysine salts, can also be prepared.

The invention also includes all possible stereoisomers of the compounds, including diastereomers and enantiomers. The invention also includes mixtures of stereoisomers in any ratio, including, but not limited to, racemic mixtures. Unless stereochemistry is explicitly indicated in a structure, the structure is intended to embrace all possible stereoisomers of the compound depicted. If stereochemistry is explicitly indicated for one portion or portions of a molecule, but not for another portion or portions of a molecule, the structure is intended to embrace all possible stereoisomers for the portion or portions where stereochemistry is not explicitly indicated.

The compounds can be administered in prodrug form. Prodrugs are derivatives of the compounds which are themselves relatively inactive, but which convert into the active compound when introduced into the subject in which they are used, by a chemical or biological process in vivo, such as an enzymatic conversion. Suitable prodrug formulations include, but are not limited to, peptide conjugates of the compounds of the invention and esters of compounds of the inventions. Further discussion of suitable prodrugs is provided in H. Bundgaard, Design of Prodrugs, New York: Elsevier, 1985; in R. Silverman, The Organic Chemistry of Drug Design and Drug Action, Boston: Elsevier, 2004; in R. L. Juliano (ed.), Biological Approaches to the Controlled Delivery of Drugs (Annals of the New York Academy of Sciences, v. 507), New York: New York Academy of Sciences, 1987; and in E. B. Roche (ed.), Design of Biopharmaceutical Properties Through Prodrugs and Analogs (Symposium sponsored by Medicinal Chemistry Section, APhA Academy of Pharmaceutical Sciences, November 1976 national meeting, Orlando, Fla.), Washington: The Academy, 1977.

Metabolites of the compounds are also embraced by the invention.

In some embodiments, the structures provided herein are also meant to include compounds which differ only in the presence of one or more isotopically enriched atoms. For example, compounds having the present structures wherein hydrogen is replaced by deuterium or tritium, or wherein carbon atom is replaced by ¹³C- or ¹⁴C-enriched carbon, are within the scope of this invention.

In other embodiments, the compounds of the present invention may also contain unnatural proportions of atomic isotopes at one or more of atoms that constitute such compounds. For example, the compounds may be radiolabeled with radioactive isotopes, such as for example deuterium (²H), tritium (³H), iodine-125 (¹²⁵I) or carbon-14 (¹⁴C). All isotopic variations of the compounds of the present invention, whether radioactive or not, are encompassed within the scope of the present invention.

“Alkyl” refers to a straight, branched, or cyclic hydrocarbon chain radical consisting solely of carbon and hydrogen atoms, containing no unsaturation, having from one to ten carbon atoms (e.g., C₁-C₁₀ alkyl). Whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated. In some embodiments, it is a C₁-C₄ alkyl group. Typical alkyl groups include, but are in no way limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl, decyl, cyclopropyl, cyclobutyl, cyclopentyl, cyclohexyl, and the like. The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, an alkyl group is optionally substituted by one or more substituents which independently are: hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, or fluoroalkyl.

“(C₁-C₄)-alkyl” is intended to embrace saturated linear, branched, or cyclic groups, or a combination of linear and/or branched and/or cyclic hydrocarbon chain and/or ring having 1 to 4 carbon atoms. Examples of “C₁-C₄ alkyl” are methyl, ethyl, n-propyl, isopropyl, cyclopropyl, n-butyl, isobutyl, sec-butyl, t-butyl, cyclobutyl, cyclopropyl-methyl, methyl-cyclopropyl. The (C₁-C₄)-alkyl is unsubstituted or is substituted by one or more of the substituents described above for an alkyl group.

“Aryl” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently: alkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, or fluoroalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively. Exemplary aralkyl radicals include but are not limited to benzyl and substituted benzyl such as-methyl-(4-fluoro)phenyl, and -ethyl-(2-hydroxy)phenyl. The aralkyl radical is connected to the compound of Formula I via a single bond to the alkyl portion of the radical.

“Benzyl” designates —CH₂-Phenyl.

“Halogen” refers to fluoro (—F), chloro (—Cl), bromo (—Br), and iodo (—I).

“Heteroaryl” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range; e.g., “5 to 18 ring atoms” means that the heteroaryl group may consist of 5 ring atoms, 6 ring atoms, etc., up to and including 18 ring atoms. An N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl,isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, a heteroaryl moiety is optionally substituted by one or more substituents which are independently: alkyl, hydroxy, halo, cyano, nitro, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, or fluoroalkyl.

Substituted heteroaryl also includes ring systems substituted with one or more oxide (—O—) substituents, such as pyridinyl N-oxides.

“Heteroaralkyl” refers to a moiety having an aryl moiety, as described herein, connected to an alkyl moiety, as described herein, wherein the connection to the remainder of the molecule is through the alkyl group. The heteroaralkyl is optionally substituted by one or more of the substituents described as suitable substituents for heteroaryl and alkyl respectively.

“Solvate” refers to a compound (e.g., a compound selected from Formula I or a pharmaceutically acceptable salt thereof) in physical association with one or more molecules of a pharmaceutically acceptable solvent. It will be understood that “a compound of Formula I” encompasses the compound of Formula I and solvates of the compound, as well as mixtures thereof. “Hydrate ” refers to a compound (e.g., a compound selected from Formula I or a pharmaceutically acceptable salt thereof) in physical association with one or more molecules of water.

In general, the nomenclature used in this Application was generated with the nomenclature software package within the ChemOffice.®. version 11.0 suite of programs by CambridgeSoft Corp (Cambridge, Mass.).

Methods of Treatment with Compounds of Formula I

This disclosure provides methods of treatment of various diseases and disorders by administering to a subject a compound of Formula I, described herein. The compounds of Formula I are tetra- and hexahydro-1H pyrido[4,3,-b]indole compounds. This section provides some nonlimiting examples of such methods of treatment.

In one aspect, the invention provides a method of treating or suppressing an oxidative stress disorder affecting normal electron flow in the cells, such as a mitochondrial disorder, a haemoglobinopathy such as thalassemia and sickle cell disease, an impaired energy processing disorder, or a disease of associated with aging, modulating one or more energy biomarkers, normalizing one or more energy biomarkers, or enhancing one or more energy biomarkers, by administering a therapeutically effective amount or effective amount of one or more compounds of Formula I:

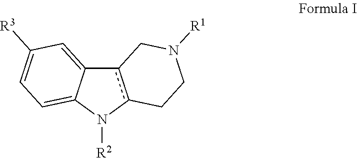

or its pharmaceutically acceptable salts, prodrugs, solvates, or hydrates thereof;
wherein R¹ is hydrogen, alkyl, aralkyl or heteroaralkyl;
R² is hydrogen, aralkyl, or heteroaralkyl;
R³ is hydrogen, alkyl, or halo; and
the bond represented by a solid line accompanied by a dotted line is a single or a double bond.

In some embodiments, the compound of Formula I is a compound having a structure of Formula I-A or Formula I-B:

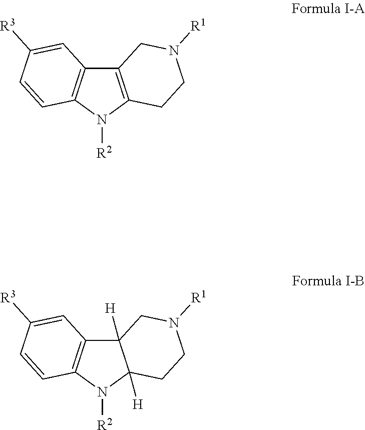

The structure of Formula I-B, as drawn, encompasses all possible stereochemical isomers. In some embodiments, the compound of Formula I-B has a structure of one of the following formulae:

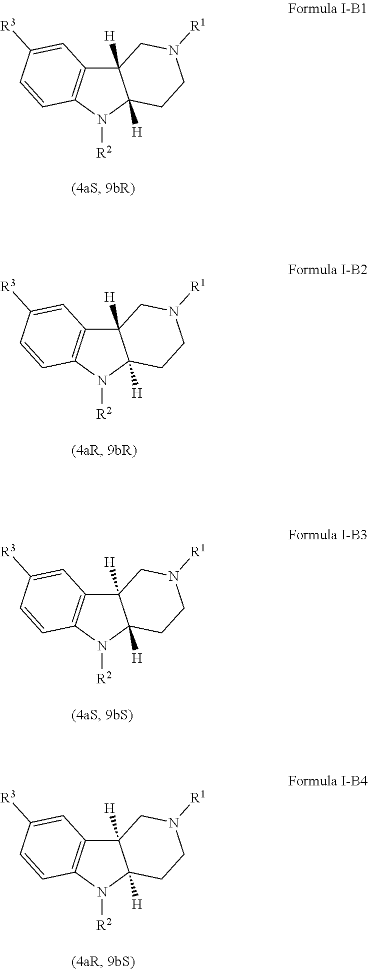

In some embodiments, the compound of Formula I-B comprises a mixture of identical chemical entities wherein the mixture has a structure of Formula I-B1. In other embodiments, the compound of Formula I-B comprises a mixture of identical chemical entities wherein the mixture has a structure of Formula I-B2. In yet other embodiments, the compound of Formula I-B comprises a mixture of identical chemical entities wherein the mixture has a structure of Formula I-B3. In further embodiments, the compound of Formula I-B comprises a mixture of identical chemical entities wherein the mixture has a structure of Formula I-B4. In various embodiments, each of the identical chemical entities of the mixture of identical chemical entities has a structure independently selected from the group consisting of Formula I-B1, Formula I-B2, Formula I-B3, and Formula I-B4. In other embodiments, the compound of Formula I-B comprises about 50%, about 60%, about 70%, about 80%, about 90%, about 95%, about 99%, or more of Formula I-B1. In other embodiments, the compound of Formula I-B comprises about 50%, about 60%, about 70%, about 80%, about 90%, about 95%, about 99%, or more of Formula I-B2. In other embodiments, the compound of Formula I-B comprises about 50%, about 60%, about 70%, about 80%, about 90%, about 95%, about 99%, or more of Formula I-B3. In other embodiments, the compound of Formula I-B comprises about 50%, about 60%, about 70%, about 80%, about 90%, about 95%, about 99%, or more of Formula I-B4. In further embodiments, the compound of Formula I-B comprises a mixture of identical chemical entities having a structure of Formula I-B4 or a structure of Formula 1-B1 in about equal proportions.

In some embodiments, R¹ is hydrogen or alkyl (including but not limited to —CH₃, —CH₂CH₃, n-propyl, isopropyl, n-butyl, tert-butyl, sec-butyl, pentyl, hexyl, and heptyl). The R¹ alkyl is unsubstituted or is substituted with one or more substituents. In other embodiments, R¹ is aralkyl, wherein nonlimiting examples include monocyclic or bicyclic aryl (including but not limited to phenyl and naphthyl) linked to alkyl (including but not limited to CH₃, —CH₂CH₃, n-propyl, isopropyl, n-butyl, sec-butyl, and pentyl). The R¹ aralkyl is unsubstituted or is substituted with one or more substituents on either the aryl or the alkyl portion of the moiety. In yet other embodiments, R¹ is heteroaralkyl (including but not limited to moncyclic or bicyclic heteroaryl) linked to alkyl (including but not limited to CH₃, —CH₂CH₃, n-propyl, isopropyl, n-butyl, sec-butyl, and pentyl). R¹ monocyclic heteroaralkyl is a monocyclic heteroaryl (including but not limited to pyridinyl, pyrimidinyl and pyrazinyl) linked to alkyl (including but not limited to CH₃, —CH₂CH₃, n-propyl, isopropyl, n-butyl, sec-butyl, and pentyl). R¹ bicyclic heteroaralkyl is a bicyclic heteroaryl (including but not limited to benzimidazolyl, quinolinyl, and indolyl) linked to alkyl (including but not limited to CH₃, —CH₂CH₃, n-propyl, isopropyl, n-butyl, sec-butyl, and pentyl). The R¹ heteroaralkyl is unsubstituted or is substituted with one or more substituents on either the aryl or the alkyl portion of the moiety.

R¹ alkyl is optionally substituted by one or more substituents chosen from the group consisting of hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, alkyl, or fluoroalkyl. R¹ aralkyl and R¹ heteroaralkyl are each optionally substituted by one or more substituents chosen from the group consisting of: alkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, or fluoroalkyl. Exemplary R¹ include but are not limited to methyl, benzyl, 2-chloro ethyl, methyl(4-methoxy)phenyl, and 3-(pyridin-3-yl)propyl.

In the compounds of Formula I, R² is hydrogen or is aralkyl (including monocyclic or bicyclic aryl (including but not limited to phenyl and naphthyl) linked to alkyl (including but not limited to CH₃, —CH₂CH₃, n-propyl, isopropyl, n-butyl, sec-butyl, and pentyl). The R² aralkyl is unsubstituted or is substituted by one or more substituents on either the aryl or the alkyl portion of the moiety. In yet other embodiments, R² is heteroaralkyl (including but not limited to monocyclic or bicyclic heteroaryl) linked to alkyl(including but not limited to CH₃, —CH₂CH₃, n-propyl, isopropyl, n-butyl, sec-butyl, and pentyl). R² monocyclic heteroaralkyl is a monocyclic heteroaryl (including but not limited to pyridinyl, pyrimidinyl and pyrazinyl) linked to alkyl (including but not limited to CH₃, —CH₂CH₃, n-propyl, isopropyl, n-butyl, sec-butyl, and pentyl). R² bicyclic heteroaralkyl is a bicyclic heteroaryl (including but not limited to benzimidazolyl, quinolinyl, and indolyl) linked to alkyl (including but not limited to CH₃, —CH₂CH₃, n-propyl, isopropyl, n-butyl, sec-butyl, and pentyl). The R² heteroaralkyl is unsubstituted or is substituted with one or more substituents on either the aryl or the alkyl portion of the moiety.

R² aralkyl and R² heteroaralkyl are each optionally substituted by one or more substituents chosen from the group consisting of :alkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, or fluoroalkyl. For example, exemplary R² include but are not limited to methyl, benzyl, 2-chloro ethyl, methyl(4-methoxy)phenyl, and 2-(6-methylpyridin-3-yl)ethyl.

In the compounds of Formula I, R³ is hydrogen or alkyl (including but not limited to —CH₃, —CH₂CH₃, n-propyl, isopropyl, n-butyl, tert-butyl, sec-butyl, pentyl, hexyl, and heptyl). The R³ alkyl is unsubstituted or is substituted by one or more substituents. In other embodiments, R³ is halo, wherein halo is chloro, fluoro, bromo or iodo.

R³ alkyl is optionally substituted by one or more substituents chosen from the group consisting of hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, alkyl, or fluoroalkyl. Exemplary R³ include but are not limited to methyl or chlorine.

In other embodiments, the compound of Formula I is the compound wherein R¹ is hydrogen, C₁-C₄-alkyl, benzyl (including unsubstituted and substituted benzyl), or a pyridinylalkyl moiety (including unsubstituted and substituted pyridinyl); R² is hydrogen, benzyl (including unsubstituted and substituted benzyl), or a pyridinylalkyl moiety (including unsubstituted and substituted pyridinyl); R³ is hydrogen, C₁-C₄-alkyl, or halogen; and a bond represented by a solid line accompanied by a dotted line is a single or double bond.

In further embodiments, the compound of Formula I is the compound wherein R¹ is hydrogen, C₁-C₄-alkyl, benzyl or 3-(pyridin-3-yl)propyl; R² is hydrogen, benzyl or 2-(6-methylpyridin-3-yl)ethyl); R³ is hydrogen, C₁-C₄-alkyl, or halogen; and a bond represented by a solid line accompanied by a dotted line is a single or double bond; or pharmaceutically acceptable salts, stereoisomers, mixtures of stereoisomers, solvates, and hydrates thereof In some embodiments of the compound of Formula I, R¹ is C₁-C₄-alkyl, benzyl or 3-(pyridin-3-yl)propyl. In other embodiments, when R¹ and/or R³ is C₁-C₄-alkyl, the C₁-C₄-alkyl is unsubstituted.

In some embodiments, the compound administered to the patient in need of such treatment is a compound of Formula I, wherein R¹ is hydrogen, methyl, ethyl or benzyl; R² is hydrogen, benzyl or 2-(6-methylpyridin-3-yl)ethyl); R³ is hydrogen, methyl, or halogen; and a bond represented by a solid line accompanied by the dotted line represents a single or double bond; or its pharmaceutically acceptable salts, stereoisomers, mixtures of stereoisomers, solvates, and hydrates thereof.

In some embodiments, the compound administered to the patient in need of such treatment is a compound of Formula I, wherein a bond represented by a solid line accompanied by the dotted line represents a double bond; or its pharmaceutically acceptable salts, solvates, and hydrates thereof.

In some embodiments, the compound administered to the patient in need of such treatment is a compound of Formula I, wherein R¹ is hydrogen, or methyl; R² is hydrogen, benzyl or 2-(6-methylpyridin-3-yl)ethyl); R³ is methyl or chlorine; and a bond represented by a solid line accompanied by the dotted line represents a single or double bond; or its pharmaceutically acceptable salts, stereoisomers, mixtures of stereoisomers, solvates, and hydrates thereof.

In some embodiments, the compound administered to the patient in need of such treatment is selected from dimebolin (2,8-dimethyl-5-(2-(6-methylpyridin-3-yl)ethyl)-2,3,4,5-tetrahydro-1H-pyrido[4,3-b]indole); 8-chloro-2-methyl-5-(2-(6-methylpyridin-3-yl)ethyl)-2,3,4,5-tetrahydro-1H-pyrido[4,3-b]indole (Dorastine); (5-benzyl-2-methyl-2,3,4,5-tetrahydro-1H-pyrido[4,3-b]indole) (Mebhydroline); 2,8-dimethyl-1,3,4,4a,5,9b-hexahydro-1H-pyrido[4,3-b]indole (including Carbidine ((+/−)cis isomers); Stobadine (a (−) cis isomer, having stereochemistry as shown in Formula I-B4); any one of (4aS, 9bR), (4aS, 9bS), (4aR, 9bR), and (4aR, 9bS) isomers; and a mixture of (4aS, 9bR), (4aS, 9bS), (4aR, 9bR), and/or (4aR, 9bS) isomers in any proportion thereof); 8-fluoro-2-(3-(pyridin-3-yl)propyl)-2,3,4,5-tetrahydro-1H-pyrido[4,3-b]indole (Gevoltroline); and 8-methyl-5-(2-(6-methylpyridin-3-yl)ethyl)-2,3,4,5-tetrahydro-1H-pyrido[4,3-b]indole; or its pharmaceutically acceptable salts, solvates, and hydrates thereof.

Compounds which are tetra- and hexahydro-1H-pyrido[4,3-b]indole derivatives are known and manifest a broad spectrum of biological activity. In the series of 2,3,4,5-tetrahydro-1H-pyrido[4,3-b]indoles the following types of activity were found: antihistamine activity (OS-DE NN 1.813 229, Dec. 6, 1968; 1.952.80, Oct. 20, 1969), central depressive and antiinflammatory activity (U.S. Pat. No. 3,718,657 Dec. 13, 1970), neuroleptic activity (Herbert C. A., Plattner S. S., Wehch W. N.—Mol. Pharm. 1980, v. 17, N 1, p. 38-42) and others. 2,3,4,4a,5,9b-hexahydro-1H-pyrido[4,3-b]indole derivatives show psychotropic (Welch W. H., Herbert C. A., Weissman A., Koe K. B. J. Med. Chem., 1986, vol. 29, No. 10, p. 2093-2099), antiaggressive, antiarrhythmic and other types of activity.

For example, Diazoline (2-methyl-5-benzyl-2,3,4,5-tetrahydro-1H-pyrido[4,3-b]indole dihydrochloride, also known as mebhydroline) (Klyuev M. A., Drugs, used in “Medical Pract.”, USSR, Moscow, “Meditzina” Publishers, 1991, p. 512) and dimebolin (2,8-dimethyl-5-(2-(6-methyl-3-pyridyl)ethyl-2,3,4,5-tetrahydro-1H-pyrido[4,3-b]indole dihydrochloride) (M. D. Mashkovsky, “Medicinal Drugs” in 2 vol. Vol. 1—12th Edition, Moscow, “Meditzina” Publishers, 1993, p. 383) (also known as “Dimebon”) as well as its closest analogue Dorastine(2-methyl-8-chloro-5-[2-(6-methyl-3-pyridyl)ethyl]-2,3,4,5-tetrahydro-1H-pyrido[4,3-b]indole dihydrochloride) (USAN and USP dictionary of drugs names (United States Adopted Names, 1961-1988, current US Pharmacopoeia and National Formular for Drugs and other nonproprietary drug names), 1989, 26th Edition., p. 196) are known as antihistamine drugs. Carbidine (dicarbine) (cis(.+/−.)-2,8-dimethyl-2,3,4,4a,5,9b-hexahydro-1H-pyrido[4,3-b]indole dihydrochloride) is a neuroleptic agent having an antidepressive effect (L. N. Yakhontov, R. G. Glushkov, Synthetic Drugs, ed. by A. G. Natradze, Moscow, “Meditzina” Publishers, 1983, p. 234-237), and its (−)isomer, Stobadine, is known as an antiarrythmic agent (Kitlova M., Gibela P., Drimal J., Bratisl. Lek. Listy, 1985, vol.84, No.5, p.542-549); Gevotroline (8-fluoro-2)(3-(3-pyridyl)-2,3,4,5-tetrahydro-1H-pyrido[4,3-b]indole dihydrochloride) is an antipsychotic and anxiolytic agent (Abou-Gharbi M., Patel U. R., Webb M. B., Moyer J. A., Ardnee T. H., J. Med. Chem., 1987, vol. 30, p. 1818-1823).

Diseases Amenable to Treatment or Suppression with Compounds and Methods of the Invention

A variety of diseases or disorders (e.g., oxidative stress disorders) are believed to be caused or aggravated by oxidative stress affecting normal electron flow in the cells, such as mitochondrial disorders, impaired energy processing disorder, and diseases of aging, such as diabetes and cancer, and can be treated or suppressed using the compounds and methods of the invention.

Such diseases include, but are not limited to, inherited mitochondrial diseases, such as Myoclonic Epilepsy with Ragged Red Fibers (MERRF), Mitochondrial Myopathy, Encephalopathy, Lactacidosis, Stroke (MELAS), Leber's Hereditary Optic Neuropathy (LHON), also referred to as Leber's Disease, Leber's Optic Atrophy (LOA), or Leber's Optic Neuropathy (LON)), chronic progressive external ophthalmoplegia (CPEO); Leigh Disease or Leigh Syndrome, Kearns-Sayre Syndrome (KSS), Friedreich's Ataxia (FRDA), Co-Enzyme Q10 (CoQ10) deficiency; other myopathies (including cardiomyopathy and encephalomyopathy), and renal tubular acidosis; motor neuron diseases; hearing and balance impairments; ataxias; other neurological diseases such as epilepsy; mood disorders such as schizophrenia and bipolar disorder; and certain age-associated diseases, particularly diseases for which CoQ10 has been proposed for treatment, such as macular degeneration, diabetes, and cancer. Mitochondrial dysfunction is also implicated in excitoxic, neuronal injury, such as that associated with seizures and ischemia. Diseases caused by energy impairment include diseases due to deprivation, poisoning or toxicity of oxygen, and qualitative or quantitative disruption in the transport of oxygen such as haemaglobionopathies (e.g., thalassemia or sickle cell anemia). Oxidative stress is suspected to be important in neurodegenerative diseases such as Motor Neuron Disease, Creutzfeldt-Jakob disease, Machado-Joseph disease, Spino-cerebellar ataxia, Multiple sclerosis(MS), and Parkinson's disease.

Spino-cerebellar ataxia is one of three main types of ataxia: cerebellar ataxia, including vestibulo-cerebellar dysfunction, spino-cerebellar dysfunction, and cerebro-cerebellar dysfunction; Sensory ataxia; and Vestibular ataxia. Examples of the diseases which are classifiable into spino-cerebellar ataxia or multiple system atrophy are hereditary olivo-ponto-cerebellar atrophy, hereditary cerebellar cortical atrophy, Friedreich's ataxia, Machado-Joseph diseases, Ramsay Hunt syndrome, hereditary dentatorubral-pallidoluysian atrophy, hereditary spastic paraplegia, Shy-Drager syndrome, cortical cerebellar atrophy, striato-nigral degeneration, Marinesco-Sjogren syndrome, alcoholic cortical cerebellar atrophy, paraneoplasic cerebellar atrophy associated with malignant tumor, toxic cerebellar atrophy caused by toxic substances, cerebellar atrophy associated with endocrine disturbance and the like.

Examples of ataxia symptoms are motor ataxia, trunk ataxia, limb ataxia and the like, autonomic disturbance such as orthostatic hypotension, dysuria, hypohidrosis, sleep apnea, orthostatic syncope and the like, stiffness of lower extremity, ocular nystagmus, oculomotor nerve disorder, pyramidal tract dysfunction, extra pyramidal symptom (postural adjustment dysfunction, muscular rigidity, akinesia, tremulus), dysphagia, lingual atrophy, posterior funiculus symptom, muscle atrophy, muscle weakness, deep hyperreflexia, sensory disturbance, scoliosis, kyphoscoliosis, foot deformans, anarthria, dementia, manic state, decreased motivation for rehabilitation and the like.

Oxidative stress is also thought to be linked to certain cardiovascular disease and also plays a role in the ischemic cascade due to oxygen reperfusion injury following hypoxia. This cascade includes both strokes and heart attacks.

Accordingly, the invention provides methods of treating or suppressing an oxidative stress disorder selected from a mitochondrial disorder, a haemoglobinopathy (e.g., thalassemia, sickle cell anemia), an impaired energy processing disorder, and a disease of aging (e.g., diabetes, cancer); modulating one or more energy biomarkers; normalizing one or more energy biomarkers; or enhancing one or more energy biomarkers, by administering a therapeutically effective amount of one or more compounds of Formula I, or pharmaceutically acceptable salts, stereoisomers, mixtures of stereoisomers, solvates, and hydrates thereof.

The invention provides for the use of a compound selected from dimebolin (8-chloro-2,8-dimethyl-5-(2-(6-methylpyridin-3-yl)ethyl)-2,3,4,5-tetrahydro-1H-pyrido[4,3-b]indole); 8-chloro-2-methyl-5-(2-(6-methylpyridin-3-yl)ethyl)-2,3,4,5-tetrahydro-1H-pyrido[4,3-b]indole; mebhydroline, 8-fluoro-2-(3-(pyridin-3-yl)propyl)-2,3,4,5-tetrahydro-1H-pyrido[4,3-b]indole, and 2,8-dimethyl-1,3,4,4a,5,9b-hexahydro-1H-pyrido[4,3-b]indole (including any one of a (4aS, 9bR), (4aS, 9bS), (4aR, 9bR), or (4aR, 9bS) isomer, or a mixture of (4aS, 9bR), (4aS, 9bS), (4aR, 9bR), and/or (4aR, 9bS) isomers in any proportion thereof); and 8-methyl-5-(2-(6-methylpyridin-3-yl)ethyl)-2,3,4,5-tetrahydro-1H-pyrido[4,3-b]indole for the treatment of an oxidative stress disorder, wherein the oxidative stress disorder is a mitochondrial disorder (including but not limited to Myoclonic Epilepsy with Ragged Red Fibers (MERRF); Mitochondrial Myopathy, Encephalopathy, Lactacidosis, and Stroke (MELAS); Leber's Hereditary Optic Neuropathy (LHON); chronic progressive external ophthalmoplegia (CPEO); Leigh Disease; Keams-Sayre Syndrome (KSS); Friedreich's Ataxia (FRDA); Co-Enzyme Q10 (CoQ10) Deficiency; Complex I Deficiency; Complex II Deficiency; Complex III Deficiency; Complex IV Deficiency; Complex V Deficiency); a haemaglobinopathy such as thalassemia and sickle cell disease; other myopathies; cardiomyopathy; encephalomyopathy; renal tubular acidosis; motor neuron diseases; hearing and balance impairments; mood disorders; schizophrenia; bipolar disorder; and age-associated diseases such as diabetes; and cancer. In a particular embodiment, the compound is dimebolin. In some embodiments, the oxidative stress disorder is not a neurodegenerative disease. In some embodiments, the oxidative stress disorder is not Huntington's disease, amyotrophic lateral sclerosis (ALS), Parkinson's disease, Alzheimer's Disease, or neuronal death mediated ocular disease. In some embodiments, the oxidative stress disorder is not Leber's Hereditary Optic Neuropathy (LHON). In some embodiments, the oxidative stress disorder is not Huntington's disease. In some embodiments, the oxidative stress disorder is not ALS. In some embodiments, the oxidative stress disorder is not Parkinson's Disease. In some embodiments, the oxidative stress disorder is not a neuronal-mediated ocular disease. In some embodiments, the oxidative stress disorder is not Alzheimer's Disease.

In some embodiments, the invention provides a method of treating or suppressing an oxidative stress disorder selected from a mitochondrial disorder, a haemaglobinopathy (e.g., thalassemia, sickle cell anemia), an impaired energy processing disorder, and a disease of aging such as diabetes and cancer; modulating one or more energy biomarkers; normalizing one or more energy biomarkers; or enhancing one or more energy biomarkers, by administering a therapeutically effective amount of dimebolin or pharmaceutically acceptable salts, solvates, and hydrates thereof.

Methods are also provided herein for treatment of an oxidative stress disorder selected from a mitochondrial disorder, a haemaglobinopathy such as thalassemia and sickle cell disease, an impaired energy processing disorder, and a disease of aging, such as diabetes and cancer modulating one or more energy biomarkers; normalizing one or more energy biomarkers; or enhancing one or more energy biomarkers, by administering a therapeutically effective amount of (8-chloro-2-methyl-5-(2-(6-methylpyridin-3-yl)ethyl)-2,3,4,5-tetrahydro-1H-pyrido[4,3-b]indole) or pharmaceutically acceptable salts, solvates, and hydrates thereof.

Alternatively, methods are provided herein for treating or suppressing an oxidative stress disorder selected from a mitochondrial disorder, a haemaglobinopathy (e.g., thalassemia or sickle cell anemia), an impaired energy processing disorder, and a disease of aging such as diabetes and cancer; modulating one or more energy biomarkers; normalizing one or more energy biomarkers; or enhancing one or more energy biomarkers, by administering a therapeutically effective amount of mebhydroline; or pharmaceutically acceptable salts, solvates, and hydrates thereof.

Methods are also provided herein for treating or suppressing an oxidative stress disorder selected from a mitochondrial disorder, a haemaglobinopathy (e.g., thalassemia, sickle cell anemia), an impaired energy processing disorder, and a disease of aging such as diabetes and cancer; modulating one or more energy biomarkers; normalizing one or more energy biomarkers; or enhancing one or more energy biomarkers, by administering a therapeutically effective amount of 8-fluoro-2-(3-(pyridin-3-yl)propyl)-2,3,4,5-tetrahydro-1H-pyrido[4,3-b]indole); or pharmaceutically acceptable salts, solvates, and hydrates thereof.

Additionally, the invention provides methods herein for treating or suppressing an oxidative stress disorder selected from a mitochondrial disorder, a haemaglobinopathy (e.g., thalassemia, sickle cell anemia), an impaired energy processing disorder, and a disease of aging, such as diabetes and cancer; modulating one or more energy biomarkers; normalizing one or more energy biomarkers; or enhancing one or more energy biomarkers, by administering a therapeutically effective amount of 2,8-dimethyl-1,3,4,4a,5,9b-hexahydro-1H-pyrido[4,3-b]indole (including Carbidine, Stobadine, and any one of (4aS, 9bR), (4aS, 9bS), (4aR, 9bR), and (4aR, 9bS) isomers, and including a mixture of (4aS, 9bR), (4aS, 9bS), (4aR, 9bR), and/or (4aR, 9bS) isomers in any proportion thereof), or pharmaceutically acceptable salts, solvates, and hydrates thereof.

The invention further provides methods for treating subjects affected with an impaired energy processing disorder due to deprivation, poisoning or toxicity of oxygen, or of qualitative or quantitative disruption in the transport of oxygen by administration of one or more compounds of Formula I or pharmaceutically acceptable salts, solvates, and hydrates thereof. In some embodiments, the disruption in transport of oxygen to tissues results in energy disruption of red cells. Diseases wherein such energy disruption of red cells occurs further include haemoglobinopathies such as sickle cell disease and thalassemia.

A. Diseases and Disorders Related to Mitochondrial Dysfunction

In one aspect of the invention, the compositions (e.g., compounds of Formula I, dimebolin, combination therapies) and methods provided herein can be used to treat or improve a wide variety of diseases or disorders caused by, or associated with, mitochondrial dysfunction. The compositions and methods provided herein may be used to treat a subject at risk for a wide variety of diseases or disorders caused by, or associated with, mitochondrial dysfunction.

Mitochondria are organelles in eukaryotic cells, popularly referred to as the “powerhouse” of the cell. One of their primary functions is oxidative phosphorylation. The molecule adenosine triphosphate (ATP) functions as an energy “currency” or energy carrier in the cell, and eukaryotic cells derive the majority of their ATP from biochemical processes carried out by mitochondria. These biochemical processes include the citric acid cycle (the tricarboxylic acid cycle, or Krebs cycle), which generates reduced nicotinamide adenine dinucleotide (NADH+H⁺) from oxidized nicotinamide adenine dinucleotide (NAD⁺), and oxidative phosphorylation, during which NADH+H⁺ is oxidized back to NAD⁺. (The citric acid cycle also reduces flavin adenine dinucleotide, or FAD, to FADH₂; FADH₂ also participates in oxidative phosphorylation.)

The electrons released by oxidation of NADH+H⁺ are shuttled down a series of protein complexes (Complex I, Complex II, Complex III, and Complex IV) known as the mitochondrial respiratory chain. These complexes are embedded in the inner membrane of the mitochondrion. Complex IV, at the end of the chain, transfers the electrons to oxygen, which is reduced to water. The energy released as these electrons traverse the complexes is used to generate a proton gradient across the inner membrane of the mitochondrion, which creates an electrochemical potential across the inner membrane. Another protein complex, Complex V (which is not directly associated with Complexes I, II, III and IV) uses the energy stored by the electrochemical gradient to convert ADP into ATP.

When cells in an organism are temporarily deprived of oxygen, anaerobic respiration is utilized until oxygen again becomes available or the cell dies. The pyruvate generated during glycolysis is converted to lactate during anaerobic respiration. The buildup of lactic acid is believed to be responsible for muscle fatigue during intense periods of activity, when oxygen cannot be supplied to the muscle cells. When oxygen again becomes available, the lactate is converted back into pyruvate for use in oxidative phosphorylation.

Mitochondrial dysfunction contributes to various disease states. Some mitochondrial diseases are due to mutations or deletions, or other genetic defects, in the mitochondrial genome. If a threshold proportion of mitochondria in the cell is defective, and if a threshold proportion of such cells within a tissue have defective mitochondria, symptoms of tissue or organ dysfunction can result. Practically any tissue can be affected, and a large variety of symptoms may be present, depending on the extent to which different tissues are involved. Some examples of mitochondrial diseases that can be treated or improved using the compositions (e.g., compounds of Formula I, dimebolin, combination therapies) and methods provided herein are Friedreich's ataxia (FRDA), Leber's Hereditary Optic Neuropathy (LHON), mitochondrial myopathy, encephalopathy, lactacidosis, and stroke (MELAS), Myoclonus Epilepsy Associated with Ragged-Red Fibers (MERRF) syndrome, and respiratory chain disorders. Most mitochondrial diseases involve children and young adults.

Friedreich's ataxia is an autosomal recessive disorder caused by decreased levels of the protein Frataxin. The disease causes the progressive loss of voluntary motor coordination (ataxia) and cardiac complications. Symptoms typically begin in childhood, and the disease progressively worsens as the patient grows older; patients eventually become wheelchair-bound due to motor disabilities.

Leber's Hereditary Optic Neuropathy (LHON) is a disease characterized by blindness which occurs on average between 27 and 34 years of age. Other symptoms may also occur, such as cardiac abnormalities and neurological complications.

Mitochondrial myopathy, encephalopathy, lactacidosis, and stroke (MELAS) can manifest itself in infants, children, or young adults. Strokes, accompanied by vomiting and seizures, are one of the most serious symptoms; it is postulated that the metabolic impairment of mitochondria in certain areas of the brain is responsible for cell death and neurological lesions, rather than the impairment of blood flow as occurs in ischemic stroke.

Myoclonus Epilepsy Associated with Ragged-Red Fibers (MERRF) syndrome is one of a group of rare muscular disorders that are called mitochondrial encephalomyopathies. Mitochondrial encephalomyopathies are disorders in which a defect in the genetic material arises from a part of the cell structure that releases energy (mitochondria). This can cause a dysfunction of the brain and muscles (encephalomyopathies). The mitochondrial defect as well as “ragged-red fibers” (an abnormality of tissue when viewed under a microscope) are always present. The most characteristic symptom of MERRF syndrome is myoclonic seizures that are usually sudden, brief, jerking, spasms that can affect the limbs or the entire body, difficulty speaking (dysarthria), optic atrophy, short stature, hearing loss, dementia, and involuntary jerking of the eyes (nystagmus) may also occur.

Leigh's disease is a rare inherited neurometabolic disorder characterized by degeneration of the central nervous system where the symptoms usually begin between the ages of 3 months to 2 years and progress rapidly. In most children, the first signs may be poor sucking ability and loss of head control and motor skills. These symptoms may be accompanied by loss of appetite, vomiting, irritability, continuous crying, and seizures. As the disorder progresses, symptoms may also include generalized weakness, lack of muscle tone, and episodes of lactic acidosis, which can lead to impairment of respiratory and kidney function. Heart problems may also occur.

Co-Enzyme Q10 Deficiency is a respiratory chain disorder, with syndromes such as myopathy with exercise intolerance and recurrent myoglobin in the urine manifested by ataxia, seizures or mental retardation and leading to renal failure (Di Mauro et al., (2005) Neuromusc. Disord., 15:311-315), childhood-onset cerebellar ataxia and cerebellar atrophy (Masumeci et al., (2001) Neurology 56:849-855 and Lamperti et al., (2003) 60:1206:1208); and infantile encephalomyopathy associated with nephrosis. Biochemical measurement of muscle homogenates of patients with CoQ10 deficiency showed severely decreased activities of respiratory chain complexes I and II+III, while complex IV (COX) was moderately decreased (Gempel et al., (2007) Brain, 130(8):2037-2044).

Complex I Deficiency or NADH dehydrogenase NADH-CoQ reductase deficiency is a respiratory chain disorder, with symptoms classified by three major forms: (1) fatal infantile multisystem disorder, characterized by developmental delay, muscle weakness, heart disease, congenital lactic acidosis, and respiratory failure; (2) myopathy beginning in childhood or in adult life, manifesting as exercise intolerance or weakness; and (3) mitochondrial encephalomyopathy (including MELAS), which may begin in childhood or adult life and consists of variable combinations of symptoms and signs, including ophthalmoplegia, seizures, dementia, ataxia, hearing loss, pigmentary retinopathy, sensory neuropathy, and uncontrollable movements.

Complex II Deficiency or Succinate dehydrogenase deficiency is a respiratory chain disorder with symptoms including encephalomyopathy and various manifestations, including failure to thrive, developmental delay, hyoptonia, lethargy, respiratory failure, ataxia, myoclonus and lactic acidosis.

Complex III Deficiency or Ubiquinone-cytochrome C oxidoreductase deficiency is a respiratory chain disorder with symptoms categorized in four major forms: (1) fatal infantile encephalomyopathy, congenital lactic acidosis, hypotonia, dystrophic posturing, seizures, and coma; (2) encephalomyopathies of later onset (childhood to adult life) various combinations of weakness, short stature, ataxia, dementia, hearing loss, sensory neuropathy, pigmentary retinopathy, and pyramidal signs; (3) myopathy, with exercise intolerance evolving into fixed weakness; and (4) infantile histiocytoid cardiomyopathy.

Complex IV Deficiency or Cytochrome C oxidase deficiency is a respiratory chain disorder with symptoms categorized in two major forms: (1) encephalomyopathy, which is typically normal for the first 6 to 12 months of life and then show developmental regression, ataxia, lactic acidosis, optic atrophy, ophthalmoplegia, nystagmus, dystonia, pyramidal signs, respiratory problems and frequent seizures; and (2) myopathy with two main variants: (a) Fatal infantile myopathy-may begin soon after birth and accompanied by hypotonia, weakness, lactic acidosis, ragged-red fibers, respiratory failure, and kidney problems: and (b) Benign infantile myopathy—may begin soon after birth and accompanied by hypotonia, weakness, lactic acidosis, ragged-red fibers, respiratory problems, but (if the child survives) followed by spontaneous improvement.

Complex V Deficiency or ATP synthase deficiency is a respiratory chain disorder including symptoms such as slow, progressive myopathy.

CPEO or Chronic Progressive External Ophthalmoplegia Syndrome is a respiratory chain disorder including symptoms such as visual myopathy, retinitis pigmentosa, or dysfunction of the central nervous system.

Kearns-Sayre Syndrome (KSS) is a mitochondrial disease characterized by a triad of features including: (1) typical onset in persons younger than age 20 years; (2) chronic, progressive, external ophthalmoplegia; and (3) pigmentary degeneration of the retina. In addition, KSS may include cardiac conduction defects, cerebellar ataxia, and raised cerebrospinal fluid (CSF) protein levels (e.g., >100 mg/dL). Additional features associated with KSS may include myopathy, dystonia, endocrine abnormalities (e.g., diabetes, growth retardation or short stature, and hypoparathyroidism), bilateral sensorineural deafness, dementia, cataracts, and proximal renal tubular acidosis.

Many respiratory chain diseases, such as those described herein, appear to be caused by defects in Complex I of the respiratory chain. Electron transfer from Complex I to the remainder of the respiratory chain is mediated by the compound coenzyme Q (also known as Ubiquinone). Oxidized coenzyme Q (CoQ^{ox} or Ubiquinone) is reduced by Complex I to reduced coenzyme Q (CoQ^{red} or Ubiquinol). The reduced coenzyme Q then transfers its electrons to Complex III of the respiratory chain (skipping over complex II), where it is re-oxidized to CoQ^{ox} (Ubiquinone). CoQ^{ox} can then participate in further iterations of electron transfer.

The methods provided herein include methods of treating a subject having, or at risk of having, an oxidative stress disorder that is a mitochondrial disease or disorder by administering one or more compounds described herein, e.g., a compound of Formula I, dimebolin, etc. In some cases, including any embodiments described herein, the oxidative stress disorder is a mitochondrial disorder selected from the group consisting of inherited mitochondrial diseases including, but not limited to: Myoclonic Epilepsy with Ragged Red Fibers (MERRF); Mitochondrial Myopathy, Encephalopathy, Lactacidosis, and Stroke (MELAS); Leber's Hereditary Optic Neuropathy (LHON); chronic progressive external ophthalmoplegia (CPEO); Leigh Disease; Kearns-Sayre Syndrome (KSS); and Friedreich's Ataxia (FRDA). In some cases, the mitochondrial disorder is Friedreich's ataxia (FRDA). In some cases, the mitochondrial disorder is Leber's Hereditary Optic Neuropathy (LHON). In some cases, the mitochondrial disorder is mitochondrial myopathy, encephalopathy, lactacidosis, and stroke (MELAS). In some cases, the mitochondrial disorder is Kearns-Sayre Syndrome (KSS). In another embodiment of the invention, the mitochondrial disorder is Myoclonic Epilepsy with Ragged Red Fibers (MERRF). In some aspects, treatment of the mitochondrial disorder may affect developmental delays observed in these disorders.

In some cases, the disclosure provides methods of treating or suppressing a mitochondrial disorder such as a mitochondrial respiratory chain disorder. In a particular embodiment, the mitochondrial respiratory chain disorder is Co-Enzyme Q10 (CoQ10) deficiency In other particular embodiments, the disorder is a defect of Complex I, Complex II, Complex III, Complex IV or Complex V, or a combination thereof.

B. Impaired Energy Processing Disorders/Diseases

The compositions (e.g., compounds of Formula I, dimebolin, combination therapies) and methods provided herein can be used to treat or improve impaired energy processing disorders due to deprivation, poisoning or toxicity of oxygen, or to qualitative or quantitative disruptions in the transport of oxygen. The methods provided herein include methods of treating a subject having, or at risk of having, an oxidative stress disorder that is an impaired energy processing disorder (e.g., haemoglobinopathy, thalassemia, sickle-cell anemia) by administering one or more compounds described herein (e.g., a compound of Formula I, dimebolin, etc.)

Haemoglobinopathies are typically caused by a genetic defect that results in abnormal structure of one of the globin chains of the hemoglobin molecule. Common haemoglobinopathies include thalassemia and sickle-cell disease. Thalassemia is an inherited autosomal recessive blood disease. In thalassemia, the genetic defect results in reduced rate of synthesis of one of the globin chains that make up hemoglobin. While thalassemia is a quantitative problem of too few globins synthesized, sickle-cell disease is a qualitative problem of synthesis of an incorrectly functioning globin. Sickle-cell disease (or sickle-cell anemia) is a blood disorder characterized by red blood cells that assume an abnormal, rigid, sickle shape. Sickling decreases the cells' flexibility and results in their restricted movement through blood vessels, depriving downstream tissues of oxygen.

In some cases, the disclosure provides a method of treating disorders caused by energy processing impairment where qualitative and/or quantitative disruptions in the function of red cells impairs the transport of oxygen to tissues. Some of these diseases include but are not limited to haemoglobinopathies (e.g., sickle cell disease/anemia, thalassemia).

The methods disclosed herein include methods of treating diseases or disorders caused by, or associated with, energy impairment due to deprivation, poisoning or toxicity of oxygen. Oxygen poisoning or toxicity is caused by high concentrations of oxygen that may be damaging to the body and increase the formation of free-radicals and other structures such as nitric oxide, peroxynitrite, and trioxidane. Normally, the body has many defense systems against such damage but at higher concentrations of free oxygen, these systems are eventually overwhelmed with time, and the rate of damage to cell membranes exceeds the capacity of systems which control or repair it. Cell damage and cell death then results.

C. Diseases and Disorders Related to Aging

The compositions (e.g., compounds of Formula I, dimebolin, combination therapies) and methods provided herein can be used to treat diseases or disorders of aging. Damage accumulation theory, also known as the free radical theory of aging, invokes random effects of free radicals produced during aerobic metabolism that cause damage to DNA, lipids and proteins and accumulate over time. The concept of free radicals playing a role in the aging process was first introduced by Himan D (1956), Aging—A theory based on free-radical and radiation chemistry J. Gerontol 11, 298-300.

According to the free radical theory of aging, the process of aging begins with oxygen metabolism (Valko et al, (2004) Role of oxygen radicals in DNA damage and cancer incidence, Mol. Cell Biochem., 266, 37-56). Even under ideal conditions some electrons “leak” from the electron transport chain. These leaking electrons interact with oxygen to produce superoxide radicals, so that under physiological conditions, about 1-3% of the oxygen molecules in the mitochondria are converted into superoxide. The primary site of radical oxygen damage from superoxide radical is mitochondrial DNA (mtDNA) (Cadenas et al., (2000) Mitochondrial free radical generation, oxidative stress and aging, Free Radic. Res, 28, 601-609). The cell repairs much of the damage done to nuclear DNA (nDNA) but mtDNA cannot be fixed. Therefore, extensive mtDNA damage accumulates over time and shuts down mitochondria causing cells to die and organism to age.

Some of the diseases associated with increasing age are cancer, diabetes mellitus, hypertension, atherosclerosis, ischemia/reperfusion injury, rheumatoid arthritis. Diseases resulting from the process of aging as a physiological decline include decreases in muscle strength, cardiopulmonary function, vision and hearing.

### Energy Biomarkers

A. Modulating Energy Biomarkers in Subjects Having, or at Risk of Having, an Oxidative Stress Disorder

In another embodiment of the invention, including any of the foregoing embodiments or other embodiments described herein, the compounds described herein are administered to subjects having, or at risk of having, a mitochondrial disorder in order to modulate one or more of various energy biomarkers, including, but not limited to, lactic acid (lactate) levels, either in whole blood, plasma, cerebrospinal fluid, or cerebral ventricular fluid; pyruvic acid (pyruvate) levels, either in whole blood, plasma, cerebrospinal fluid, or cerebral ventricular fluid; lactate/pyruvate ratios, either in whole blood, plasma, cerebrospinal fluid, or cerebral ventricular fluid; phosphocreatine levels, NADH (NADH+H+) or NADPH (NADPH+H+) levels; NAD or NADP levels; ATP levels; reduced coenzyme Q (CoQred) levels; oxidized coenzyme Q (CoQox) levels; total coenzyme Q (CoQtot) levels; oxidized cytochrome C levels; reduced cytochrome C levels; oxidized cytochrome C/reduced cytochrome C ratio; acetoacetate levels; beta-hydroxy butyrate levels; acetoacetate/beta-hydroxy butyrate ratio; 8-hydroxy-2′-deoxyguanosine (8-OHdG) levels; levels of reactive oxygen species; oxygen consumption (VO₂), carbon dioxide output (VCO₂), respiratory quotient (VCO₂/VO₂), and to modulate exercise intolerance (or conversely, modulate exercise tolerance) and to modulate anaerobic threshold. Energy biomarkers can be measured in whole blood, plasma, cerebrospinal fluid, cerebroventricular fluid, arterial blood, venous blood, or any other body fluid, body gas, or other biological sample useful for such measurement. In one embodiment, the levels are modulated to a value within about 2 standard deviations of the value in a healthy subject. In another embodiment, the levels are modulated to a value within about 1 standard deviation of the value in a healthy subject. In another embodiment, the levels in a subject are changed by at least about 10% above or below the level in the subject prior to modulation. In another embodiment, the levels are changed by at least about 20% above or below the level in the subject prior to modulation. In another embodiment, the levels are changed by at least about 30% above or below the level in the subject prior to modulation. In another embodiment, the levels are changed by at least about 40% above or below the level in the subject prior to modulation. In another embodiment, the levels are changed by at least about 50% above or below the level in the subject prior to modulation. In another embodiment, the levels are changed by at least about 75% above or below the level in the subject prior to modulation. In another embodiment, the levels are changed by at least about 100% above or at least about 90% below the level in the subject prior to modulation.

In another embodiment, including any of the foregoing embodiments or other embodiments described herein, the subject or subjects in which a method of treating or suppressing an oxidative stress disorder, modulating one or more energy biomarkers, normalizing one or more energy biomarkers, or enhancing one or more energy biomarkers is performed is/are selected from the group consisting of subjects undergoing strenuous or prolonged physical activity; subjects with chronic energy problems; subjects with chronic respiratory problems; pregnant females; pregnant females in labor; neonates; premature neonates; subjects exposed to extreme environments; subjects exposed to hot environments; subjects exposed to cold environments; subjects exposed to environments with lower-than-average oxygen content; subjects exposed to environments with higher-than-average carbon dioxide content; subjects exposed to environments with higher-than-average levels of air pollution; airline travelers; flight attendants; subjects at elevated altitudes; subjects living in cities with lower-than-average air quality; subjects working in enclosed environments where air quality is degraded; subjects with lung diseases; subjects with lower-than-average lung capacity; tubercular patients; lung cancer patients; emphysema patients; cystic fibrosis patients; subjects recovering from surgery; subjects recovering from illness; elderly subjects; elderly subjects experiencing decreased energy; subjects suffering from chronic fatigue; subjects suffering from chronic fatigue syndrome; subjects undergoing acute trauma; subjects in shock; subjects requiring acute oxygen administration; subjects requiring chronic oxygen administration; or other subjects with acute, chronic, or ongoing energy demands who can benefit from enhancement of energy biomarkers.

B. Clinical Assessment of Mitochondrial Dysfunction and Efficacy of Therapy

Several readily measurable clinical markers are used to assess the metabolic state of subjects with mitochondrial disorders or impaired energy processing disorders. These markers can also be used as indicators of the efficacy of a given therapy, as the level of a marker is moved from the pathological value to the healthy value. These clinical markers include, but are not limited to, one or more of the previously discussed energy biomarkers, such as lactic acid (lactate) levels, either in whole blood, plasma, cerebrospinal fluid, or cerebral ventricular fluid; pyruvic acid (pyruvate) levels, either in whole blood, plasma, cerebrospinal fluid, or cerebral ventricular fluid; lactate/pyruvate ratios, either in whole blood, plasma, cerebrospinal fluid, or cerebral ventricular fluid; phosphocreatine levels, NADH (NADH+H⁺) or NADPH (NADPH+H⁺) levels; NAD or NADP levels; ATP levels; anaerobic threshold; reduced coenzyme Q (CoQ^{red}) levels; oxidized coenzyme Q (CoQ^{ox}) levels; total coenzyme Q (CoQ^{tot}) levels; oxidized cytochrome C levels; reduced cytochrome C levels; oxidized cytochrome C/reduced cytochrome C ratio; acetoacetate levels, β-hydroxy butyrate levels, acetoacetate/β-hydroxy butyrate ratio, 8-hydroxy-2′-deoxyguanosine (8-OHdG) levels; levels of reactive oxygen species; and levels of oxygen consumption (VO₂), levels of carbon dioxide output (VCO₂), and respiratory quotient (VCO₂/VO₂). Several of these clinical markers are measured routinely in exercise physiology laboratories, and provide convenient assessments of the metabolic state of a subject. In one embodiment of the invention, the level of one or more energy biomarkers in a patient suffering from a mitochondrial disease, such as Friedreich's ataxia, Leber's hereditary optic neuropathy, MELAS, or KSS, is improved to within two standard deviations of the average level in a healthy subject. In another embodiment of the invention, the level of one or more of these energy biomarkers in a patient suffering from a mitochondrial disease, such as Friedreich's ataxia, Leber's hereditary optic neuropathy, MELAS, or KSS is improved to within one standard deviation of the average level in a healthy subject. Exercise intolerance can also be used as an indicator of the efficacy of a given therapy, where an improvement in exercise tolerance (i.e., a decrease in exercise intolerance) indicates efficacy of a given therapy.

Several metabolic biomarkers have already been used to evaluate efficacy of CoQ10, and these metabolic biomarkers can be monitored as energy biomarkers for use in the methods of the current invention. Pyruvate, a product of the anaerobic metabolism of glucose, is removed by reduction to lactic acid in an anaerobic setting or by oxidative metabolism, which is dependent on a functional mitochondrial respiratory chain. Dysfunction of the respiratory chain may lead to inadequate removal of lactate and pyruvate from the circulation and elevated lactate/pyruvate ratios are observed in mitochondrial cytopathies (see Scriver C R, The metabolic and molecular bases of inherited disease, 7th ed., New York: McGraw-Hill, Health Professions Division, 1995; and Munnich et al., J. Inherit. Metab. Dis. 15(4):448-55 (1992)). Blood lactate/pyruvate ratio (Chariot et al., Arch. Pathol. Lab. Med. 118(7):695-7 (1994)) is, therefore, widely used as a noninvasive test for detection of mitochondrial cytopathies (see again Scriver C R, The metabolic and molecular bases of inherited disease, 7th ed., New York: McGraw-Hill, Health Professions Division, 1995; and Munnich et al., J. Inherit. Metab. Dis. 15(4):448-55 (1992)) and toxic mitochondrial myopathies (Chariot et al., Arthritis Rheum. 37(4):583-6 (1994)). Changes in the redox state of liver mitochondria can be investigated by measuring the arterial ketone body ratio (acetoacetate/3-hydroxybutyrate: AKBR) (Ueda et al., J. Cardiol. 29(2):95-102 (1997)). Urinary excretion of 8-hydroxy-2′-deoxyguanosine (8-OHdG) often has been used as a biomarker to assess the extent of repair of ROS-induced DNA damage in both clinical and occupational settings (Erhola et al, FEBS Lett. 409(2):287-91 (1997); Honda et al., Leuk. Res. 24(6):461-8 (2000); Pilger et al., Free Radic. Res. 35(3):273-80 (2001); Kim et al. Environ Health Perspect 112(6):666-71 (2004)).

Magnetic resonance spectroscopy (MRS) has been useful in the diagnoses of mitochondrial cytopathy by demonstrating elevations in cerebrospinal fluid (CSF) and cortical white matter lactate using proton MRS (1H-MRS) (Kaufmann et al., Neurology 62(8):1297-302 (2004)). Phosphorous MRS (31P-MRS) has been used to demonstrate low levels of cortical phosphocreatine (PCr) (Matthews et al., Ann. Neurol. 29(4):435-8 (1991)), and a delay in PCr recovery kinetics following exercise in skeletal muscle (Matthews et al., Ann. Neurol. 29(4):435-8 (1991); Barbiroli et al., J. Neurol. 242(7):472-7 (1995); Fabrizi et al., J. Neurol. Sci. 137(1):20-7 (1996)). A low skeletal muscle PCr has also been confirmed in patients with mitochondrial cytopathy by direct biochemical measurements.

Exercise testing is particularly helpful as an evaluation and screening tool in mitochondrial myopathies. One of the hallmark characteristics of mitochondrial myopathies is a reduction in maximal whole body oxygen consumption (VO₂max) (Taivassalo et al., Brain 126(Pt 2):413-23 (2003)). Given that VO₂max is determined by cardiac output (Qc) and peripheral oxygen extraction (arterial-venous total oxygen content) difference, some mitochondrial cytopathies affect cardiac function where delivery can be altered; however, most mitochondrial myopathies show a characteristic deficit in peripheral oxygen extraction (A-VO₂ difference) and an enhanced oxygen delivery (hyperkinetic circulation) (Taivassalo et al., Brain 126(Pt 2):413-23 (2003)). This can be demonstrated by a lack of exercise induced deoxygenation of venous blood with direct AV balance measurements (Taivassalo et al, Ann. Neurol. 51(1):38-44 (2002)) and non-invasively by near infrared spectroscopy (Lynch et al., Muscle Nerve 25(5):664-73 (2002); van Beekvelt et al., Ann. Neurol. 46(4):667-70 (1999)).

Several of these energy biomarkers are discussed in more detail as follows. It should be emphasized that, while certain energy biomarkers are discussed and enumerated herein, the invention is not limited to modulation, normalization or enhancement of only these enumerated energy biomarkers.

Lactic acid (lactate) levels: Mitochondrial dysfunction typically results in abnormal levels of lactic acid, as pyruvate levels increase and pyruvate is converted to lactate to maintain capacity for glycolysis. Mitochondrial dysfunction can also result in abnormal levels of NADH+H⁺, NADPH+H⁺, NAD, or NADP, as the reduced nicotinamide adenine dinucleotides are not efficiently processed by the respiratory chain. Lactate levels can be measured by taking samples of appropriate bodily fluids such as whole blood, plasma, or cerebrospinal fluid. Using magnetic resonance, lactate levels can be measured in virtually any volume of the body desired, such as the brain.

Measurement of cerebral lactic acidosis using magnetic resonance in MELAS patients is described in Kaufmann et al., Neurology 62(8):1297 (2004). Values of the levels of lactic acid in the lateral ventricles of the brain are presented for two mutations resulting in MELAS, A3243G and A8344G. Whole blood, plasma, and cerebrospinal fluid lactate levels can be measured by commercially available equipment such as the YSI 2300 STAT Plus Glucose & Lactate Analyzer (YSI Life Sciences, Ohio).

NAD, NADP, NADH and NADPH levels: Measurement of NAD, NADP, NADH (NADH+H⁺) or NADPH (NADPH+H⁺) can be measured by a variety of fluorescent, enzymatic, or electrochemical techniques, e.g., the electrochemical assay described in US 2005/0067303.

Oxygen consumption (vO₂ or VO₂), carbon dioxide output (vCO₂ or VCO₂), and respiratory quotient (VCO₂/VO₂): VO₂ is usually measured either while resting (resting VO₂) or at maximal exercise intensity (VO₂ max). Optimally, both values will be measured. However, for severely disabled patients, measurement of VO₂ max may be impractical. Measurement of both forms of VO₂ is readily accomplished using standard equipment from a variety of vendors, e.g. Korr Medical Technologies, Inc. (Salt Lake City, Utah). VCO₂ can also be readily measured, and the ratio of VCO₂ to VO₂ under the same conditions (VCO₂/VO₂, either resting or at maximal exercise intensity) provides the respiratory quotient (RQ).

Oxidized Cytochrome C, reduced Cytochrome C, and ratio of oxidized Cytochrome C to reduced Cytochrome C: Cytochrome C parameters, such as oxidized cytochrome C levels (Cyt Cₒₓ), reduced cytochrome C levels (Cyt C_{red}), and the ratio of oxidized cytochrome C/reduced cytochrome C ratio (Cyt Cₒₓ)/(Cyt C_{red}), can be measured by in vivo near infrared spectroscopy. See, e.g., Rolfe, P., “In vivo near-infrared spectroscopy,” Ann. Rev. Biomed. Eng. 2:715-54 (2000), and Strangman et al, “Non-invasive neuroimaging using near-infrared light” Biol. Psychiatry 52:679-93 (2002).

Exercise tolerance/Exercise intolerance: Exercise intolerance is defined as “the reduced ability to perform activities that involve dynamic movement of large skeletal muscles because of symptoms of dyspnea or fatigue” (Pi\ddot{n}a et al, Circulation 107:1210 (2003)). Exercise intolerance is often accompanied by myoglobinuria, due to breakdown of muscle tissue and subsequent excretion of muscle myoglobin in the urine. Various measures of exercise intolerance can be used, such as time spent walking or running on a treadmill before exhaustion, time spent on an exercise bicycle (stationary bicycle) before exhaustion, and the like. Treatment with the compounds or methods of the invention can result in about a 10% or greater improvement in exercise tolerance (for example, about a 10% or greater increase in time to exhaustion, e.g., from 10 minutes to 11 minutes), about a 20% or greater improvement in exercise tolerance, about a 30% or greater improvement in exercise tolerance, about a 40% or greater improvement in exercise tolerance, about a 50% or greater improvement in exercise tolerance, about a 75% or greater improvement in exercise tolerance, or about a 100% or greater improvement in exercise tolerance. While exercise tolerance is not, strictly speaking, an energy biomarker, for the purposes of the invention, modulation, normalization, or enhancement of energy biomarkers includes modulation, normalization, or enhancement of exercise tolerance.

Similarly, tests for normal and abnormal values of pyruvic acid (pyruvate) levels, lactate/pyruvate ratio, ATP levels, anaerobic threshold, reduced coenzyme Q (CoQ^{red}) levels, oxidized coenzyme Q (CoQ^{ox}) levels, total coenzyme Q (CoQ^{tot}) levels, oxidized cytochrome C levels, reduced cytochrome C levels, oxidized cytochrome C/reduced cytochrome C ratio, acetoacetate levels, β-hydroxy butyrate levels, acetoacetate/β-hydroxy butyrate ratio, 8-hydroxy-2′-deoxyguanosine (8-OHdG) levels, and levels of reactive oxygen species are known in the art and can be used to evaluate efficacy of the compounds and methods of the invention. (For the purposes of the invention, modulation, normalization, or enhancement of energy biomarkers includes modulation, normalization, or enhancement of anaerobic threshold.)

C. Biomarker Correlation to Disease State or Improvement Thereof

As is shown in Table 1, the effect that various dysfunctions can have on biochemical and energy biomarkers is illustrated. It also indicates the physical effect (such as a disease symptom or other effect of the dysfunction) typically associated with a given dysfunction. It should be noted that any of the energy biomarkers listed in the table, in addition to energy biomarkers enumerated elsewhere, can also be modulated, enhanced, or normalized by the compounds and methods of the invention. RQ=respiratory quotient; BMR=basal metabolic rate; HR (CO)=heart rate (cardiac output); T=body temperature (preferably measured as core temperature); AT=anaerobic threshold; pH=blood pH (venous and/or arterial).

TABLE 1

| Site of | | Measurable Energy | |
|---|---|---|---|
| Dysfunction | Biochemical Event | Biomarker | Physical Effect |
| Respiratory | ↑ NADH | Δ lactate, Δ lactate: pyruvate | Metabolic |
| Chain | | ratio; and | dyscrasia & fatigue |
| | | Δ acetoacetate: β-hydroxy | |
| | | butyrate ratio | |
| Respiratory | ↓ H⁺ gradient | Δ ATP | Organ dependent |
| Chain | | | dysfunction |
| Respiratory | ↓ Electron flux | Δ VO₂, RQ, BMR, ΔT, AT, | Metabolic |
| Chain | | pH | dyscrasia & fatigue |
| Mitochondria & | ↓ ATP, ↓ VO₂ | Δ Work, ΔHR (CO) | Exercise |
| cytosol | | | intolerance |
| Mitochondria & | ↓ ATP | Δ PCr | Exercise |
| cytosol | | | intolerance |
| Respiratory | ↓ Cyt C_{Ox/Red} | Δ λ~700-900 nM (Near | Exercise |
| Chain | | Infrared Spectroscopy) | intolerance |
| Intermediary | ↓ Catabolism | Δ C¹⁴-Labeled substrates | Metabolic |
| metabolism | | | dyscrasia & fatigue |
| Respiratory | ↓ Electron flux | Δ Mixed Venous VO₂ | Metabolic |
| Chain | | | dyscrasia & fatigue |
| Mitochondria & | ↑ Oxidative stress | Δ Tocopherol & | Uncertain |
| cytosol | | Tocotrienols, CoQ10_{,} | |
| | | docosahexanoic acid | |
| Mitochondria & | ↑ Oxidative stress | Δ Glutathione_{red} | Uncertain |
| cytosol | | | |
| Mitochondria & | Nucleic acid | [symbol] 8-hydroxy 2-deoxy | Uncertain |
| cytosol | oxidation | guanosine | |
| Mitochondria & | Lipid oxidation | Δ Isoprostane(s), eicasanoids | Uncertain |
| cytosol | | | |
| Cell membranes | Lipid oxidation | Δ Ethane (breath) | Uncertain |
| Cell membranes | Lipid oxidation | Δ Malondialdehyde | Uncertain |

Treatment of a subject afflicted by a mitochondrial disease in accordance with the methods of the invention may result in the inducement of a reduction or alleviation of symptoms in the subject, e.g., to halt the further progression of the disorder.

Partial or complete suppression of the mitochondrial disease can result in a lessening of the severity of one or more of the symptoms that the subject would otherwise experience. For example, partial suppression of MELAS could result in reduction in the number of stroke-like or seizure episodes suffered.

Any one or any combination of the energy biomarkers described herein provides conveniently measurable benchmarks by which to gauge the effectiveness of treatment or suppressive therapy. Additionally, other energy biomarkers are known to those skilled in the art and can be monitored to evaluate the efficacy of treatment or suppressive therapy.

D. Use of Compounds for Modulation of Energy Biomarker(s)

In addition to monitoring energy biomarkers to assess the status of treatment or suppression of mitochondrial diseases or impaired energy processing disorders, the compounds of the invention can be used in subjects to modulate one or more energy biomarkers. Modulation of energy biomarkers can be done to normalize energy biomarkers in a subject, or to enhance energy biomarkers in a subject.

Normalization of one or more energy biomarkers is defined as either restoring the level of one or more such energy biomarkers to normal or near-normal levels in a subject whose levels of one or more energy biomarkers show pathological differences from normal levels (i.e., levels in a healthy subject), or to change the levels of one or more energy biomarkers to alleviate pathological symptoms in a subject. Depending on the nature of the energy biomarker, such levels may show measured values either above or below a normal value. For example, a pathological lactate level is typically higher than the lactate level in a normal (i.e., healthy) person, and a decrease in the level may be desirable. A pathological ATP level is typically lower than the ATP level in a normal (i.e., healthy) person, and an increase in the level of ATP may be desirable. Accordingly, normalization of energy biomarkers can involve restoring the level of energy biomarkers to within about at least two standard deviations of normal in a subject, more preferably to within about at least one standard deviation of normal in a subject, to within about at least one-half standard deviation of normal, or to within about at least one-quarter standard deviation of normal.

Enhancement of the level of one or more energy biomarkers is defined as changing the extant levels of one or more energy biomarkers in a subject to a level which provides beneficial or desired effects for the subject. For example, a person undergoing strenuous effort or prolonged vigorous physical activity, such as mountain climbing, could benefit from increased ATP levels or decreased lactate levels. As described above, normalization of energy biomarkers may not achieve the optimum state for a subject with a mitochondrial disease, and such subjects can also benefit from enhancement of energy biomarkers. Examples of subjects who could benefit from enhanced levels of one or more energy biomarkers include, but are not limited to, subjects undergoing strenuous or prolonged physical activity, subjects with chronic energy problems, or subjects with chronic respiratory problems. Such subjects include, but are not limited to, pregnant females, particularly pregnant females in labor; neonates, particularly premature neonates; subjects exposed to extreme environments, such as hot environments (temperatures routinely exceeding about 85-86 degrees Fahrenheit or about 30 degrees Celsius for about 4 hours daily or more), cold environments (temperatures routinely below about 32 degrees Fahrenheit or about 0 degrees Celsius for about 4 hours daily or more), or environments with lower-than-average oxygen content, higher-than-average carbon dioxide content, or higher-than-average levels of air pollution (airline travelers, flight attendants, subjects at elevated altitudes, subjects living in cities with lower-than-average air quality, subjects working in enclosed environments where air quality is degraded); subjects with lung diseases or lower-than-average lung capacity, such as tubercular patients, lung cancer patients, emphysema patients, and cystic fibrosis patients; subjects recovering from surgery or illness; elderly subjects, including elderly subjects experiencing decreased energy; subjects suffering from chronic fatigue, including chronic fatigue syndrome; subjects undergoing acute trauma; subjects in shock; subjects requiring acute oxygen administration; subjects requiring chronic oxygen administration; or other subjects with acute, chronic, or ongoing energy demands who can benefit from enhancement of energy biomarkers.

Accordingly, when an increase in a level of one or more energy biomarkers is beneficial to a subject, enhancement of the one or more energy biomarkers can involve increasing the level of the respective energy biomarker or energy biomarkers to about at least one-quarter standard deviation above normal, about at least one-half standard deviation above normal, about at least one standard deviation above normal, or about at least two standard deviations above normal. Alternatively, the level of the one or more energy biomarkers can be increased by about at least 10% above the subject's level of the respective one or more energy biomarkers before enhancement, by about at least 20% above the subject's level of the respective one or more energy biomarkers before enhancement, by about at least 30% above the subject's level of the respective one or more energy biomarkers before enhancement, by about at least 40% above the subject's level of the respective one or more energy biomarkers before enhancement, by about at least 50% above the subject's level of the respective one or more energy biomarkers before enhancement, by about at least 75% above the subject's level of the respective one or more energy biomarkers before enhancement, or by about at least 100% above the subject's level of the respective one or more energy biomarkers before enhancement.

When a decrease in a level of one or more energy biomarkers is desired to enhance one or more energy biomarkers, the level of the one or more energy biomarkers can be decreased by an amount of about at least one-quarter standard deviation of normal in a subject, decreased by about at least one-half standard deviation of normal in a subject, decreased by about at least one standard deviation of normal in a subject, or decreased by about at least two standard deviations of normal in a subject. Alternatively, the level of the one or more energy biomarkers can be decreased by about at least 10% below the subject's level of the respective one or more energy biomarkers before enhancement, by about at least 20% below the subject's level of the respective one or more energy biomarkers before enhancement, by about at least 30% below the subject's level of the respective one or more energy biomarkers before enhancement, by about at least 40% below the subject's level of the respective one or more energy biomarkers before enhancement, by about at least 50% below the subject's level of the respective one or more energy biomarkers before enhancement, by about at least 75% below the subject's level of the respective one or more energy biomarkers before enhancement, or by about at least 90% below the subject's level of the respective one or more energy biomarkers before enhancement.

Treating Subjects After, or in Combination with, Genetic Screening

Because many of the mitochondrial diseases or disorders are inherited, genetic screening can be used to identify subjects at risk for such diseases or disorders. The compounds and methods described herein can then be administered to asymptomatic subjects at risk of developing the clinical symptoms of the disease, in order to suppress the appearance of any adverse symptoms. Thus, the methods provided herein include steps to test or screen a subject for a genetic defect that can cause an oxidative stress disorder (e.g., mitochondrial disease) followed by, or in combination with, administering to the subject one or more compounds described herein (e.g., compounds of Formula I, dimebolin), either alone or in combination with another therapy (e.g., antioxidants, erythropoietin (“EPO”, including biosimilars, mutants, and mimetics thereof), Idebenone, and/or MitoQ).

In order to test for Friedreich's Ataxia, a subject may be tested for mutations in the gene FXN, which encodes the Frataxin protein. A subject testing positive for mutations in the gene FXN may be treated with one or more compounds described herein (e.g., compounds of Formula I, dimebolin, etc.). In some cases, the one or more compounds may be combined with one or more other compounds that may be effective against Friedreich's Ataxia, or other mitochondrial disorder, such as Idebenone or MitoQ. In some cases, a subject testing positive for mutations in the gene FXN may be treated with a compound of Formula I in combination with idebenone and/or EPO (including biosimilars, mutants, and mimetics thereof). In some cases, a subject testing positive for mutations in the gene FXN may be treated with a compound of Formula I in combination with EPO (including biosimilars, mutants, and mimetics thereof). In some cases, a subject testing positive for mutations in the gene FXN may be treated with a compound of Formula I in combination with antioxidants.

In some cases, a subject's predisposition for Coenzyme Q10 (CoQ10) Deficiency is analyzed by testing for genetic defects or mutations in one or more of the following genes: mitochondrial parahydroxybenzoid-polyprenyltransferase (COQ2); APTX; decaprenyl diphosphate synthase subunit-2 gene (PDSS2); PDSS1; and CABC1. A subject testing positive for one or more genetic defects that cause CoQ10 Deficiency may be treated with one or more compounds described herein (e.g., compounds of Formula I, dimebolin, etc.). In some cases, the one or more compounds may be combined with one or more other compounds known to be effective against CoQ10 Deficiency, or other mitochondrial disorder. In some cases, a subject testing positive for genetic defects that cause CoQ10 Deficiency may be treated with a compound of Formula I in combination with oral CoQ10. In some cases, a subject testing positive for genetic defects that cause CoQ10 Deficiency may be treated with a compound of Formula I in combination with oral CoQ10, EPO (including biosimilars, mutants, and mimetics thereof), and/or antioxidants.

Similarly a subject may be tested for mutations in one or more of the following genes in order to determine the subject's predisposition for Complex I Deficiency: a gene (mitochondrial or nuclear) encoding any subunit of Human complex I (NADH-ubiquinone reductase), NDUFV1, NDUFV2, NDUFS1, NDUFS2, NDUFS3, NDUFS4, NDUFS6, NDUFS7, NDUFS8, NDUFA2, NDUFA11, B17.2L, HRPAP20, C20ORF7, NDUFA1. Similarly a subject may be tested for mutations in one or more of the following genes in order to determine the subject's predisposition for Complex I Deficiency: a gene encoding any component of complex I, MTND1, MTND2, MTND3, MTND4, MTND51, MTND6, or MTTS2.

In order to test a subject's predisposition for Complex II Deficiency, the subject may be tested for genetic defects in the gene encoding succinate dehydrogenase (SDHA). In order to test a subject's predisposition for Mitochondrial Complex III Deficiency, the subject may be tested for genetic defects in a gene associated with III Deficiency (e.g., BCS1L, UQCRB, or UQCRQ). In order to test a subject's predisposition for Complex IV Deficiency, the subject may be tested for genetic defects in a gene associated with Complex IV Deficiency (e.g., one or more mitochondrial COX genes such as MTCO1, MTCO2, MTCO3; mitochondrial tRNA(ser) (MTTS1) and tRNA(leu) (MTTL1); or nuclear genes such as COX10, COX6B1, SCO1, SCO2 and FASTKD2). In order to test a subject's predisposition for Complex V Deficiency, the subject may be tested for genetic defects in a gene associated with Complex V Deficiency (e.g., a gene encoding mitochondrial ATP synthase F1 complex assembly factor-2 (ATPAF2), or TMEM70).

In order to test for Leber's Hereditary Optic Neuropathy (LHON), a subject may be tested for mutations in a gene encoding the NADH dehydrogenase protein, which is involved in the oxidative phosphorylation (e.g., MT-ND1, MT-ND4, MT-ND4L, and MT-ND6). A subject testing positive for mutations in one or more of such genes may be treated with one or more compounds described herein (e.g., compounds of Formula I, dimebolin, etc.). In some cases, the one or more compounds may be combined with one or more other compounds known to be effective against LHON, or other mitochondrial disorder. In some cases, a subject testing positive for mutations in a gene encoding NADH dehydrogenase protein (e.g., MT-ND1, MT-ND4, MT-ND4L, and MT-ND6) may be treated with a compound of Formula I in combination with one or more of the following: Brimonidine; Minocycline; Idebenone; Curcumin; glutathione; Near infrared light treatment; and Viral vector techniques. In other cases, a subject testing positive for for mutations in a gene encoding NADH dehydrogenase protein (e.g., MT-ND1, MT-ND4, MT-ND4L, and MT-ND6) may be treated with a compound of Formula I in combination with EPO (including biosimilars, mutants, and mimetics thereof) and/or antioxidants.

In order to test for Mitochondrial Myopathy, Encephalopathy, Lactacidosis, Stroke (MELAS) a subject may be tested for mutations or genetic defects associated with MELAS (e.g., A3243G or A8344G mitochondrial mutations). In some cases, a subject is tested for mutations in one or more of the following genes: MT-ND1, MT-ND5, MT-TH, MT-TL1, and MT-TV. In some cases, the subject may be tested for mutations in genes encoding mitochondrial tRNA. A subject testing positive for mutations in one or more of such genes may be treated with one or more compounds described herein (e.g., compounds of Formula I, dimebolin, etc.). In some cases, the one or more compounds may be combined with one or more other compounds known to be effective against MELAS, or other mitochondrial disorder. In some cases, a subject testing positive for a genetic defect associated with MELAS, or mutations in a MT-ND1, MT-ND5, MT-TH, MT-TL1, and/or MT-TV, may be treated with a compound of Formula I in combination with one or more of the following: CoQ10, Riboflavin, L-arginine, resveratrol, and SIRT1 activators. In some cases, a subject testing positive for a genetic defect associated with MELAS, or mutations in a MT-ND1, MT-ND5, MT-TH, MT-TL1, and/or MT-TV, may be treated with a compound of Formula I in combination with CoQ10, EPO (including biosimilars, mutants, and mimetics thereof), and/or antioxidants.

In order to test Myoclonic Epilepsy with Ragged Red Fibers (MERRF); a subject may be tested for mutations in one or more mitochondrial genes encoding tRNA-lys. For example, a subject may be tested for mutations in one or more of the following genes: MT-TK, MT-TL1, MT-TH, MT-TS1, MT-TS2, and MT-TF A subject testing positive for mutations in one or more of such genes may be treated with one or more compounds described herein (e.g., compounds of Formula I, dimebolin, etc.). In some cases, the one or more compounds may be combined with one or more other compounds known to be effective against MERRF, or other mitochondrial disorder. In some cases, a subject testing positive for a genetic defect associated with MERRF, or for mutations in: MT-TK, MT-TL1, MT-TH, MT-TS1, MT-TS2, and/or MT-TF, may be treated with a compound of Formula I in combination with one or more of the following: CoQ10 and L-Carnitine. In some cases, a subject testing positive for a genetic defect associated with MERRF, or mutations in: MT-TK, MT-TL1, MT-TH, MT-TS1, MT-TS2, and/or MT-TF, may be treated with a compound of Formula I in combination with EPO (including biosimilars, mutants, and mimetics thereof) and/or antioxidants.

In order to test for chronic progressive external ophthalmoplegia (CPEO); a subject may be tested for specific deletions or mutations in mitochondrial DNA. A subject testing positive for mutations or deletions in regions of mitochondrial DNA known to be associated with CPEO, may be treated with one or more compounds described herein (e.g., compounds of Formula I, dimebolin, etc.). In some cases, the one or more compounds may be combined with one or more other compounds known to be effective against CPEO, or other mitochondrial disorder (e.g., Idebenone, EPO (including biosimilars, mutants, and mimetics thereof), and/or antioxidants).

In order to test for Kearns-Sayre Syndrome (KSS), a subject may be tested for specific deletions mitochondrial DNA (e.g., 4,977 base-pair deletion in the mitochondrial DNA). A subject testing positive for deletions in regions of mitochondrial DNA known to be associated with KSS, may be treated with one or more compounds described herein (e.g., compounds of Formula I, dimebolin, etc.). In some cases, the one or more compounds may be combined with one or more other compounds known to be effective against KSS, or other mitochondrial disorder (e.g., Idebenone, EPO (including biosimilars, mutants, and mimetics thereof), and/or antioxidants).

In order to test for Leigh Disease, a subject may be tested for one or more of the following: mutations in mitochondrial DNA (mtDNA), mutations in nuclear DNA (e.g., SURF1, COX). A subject testing positive for a genetic defect associated with Leigh Disease may be treated with one or more compounds described herein (e.g., compounds of Formula I, dimebolin, etc.). In some cases, the one or more compounds may be combined with one or more other compounds known to be effective against Leigh Disease (e.g., thiamin) or other mitochondrial disorder (e.g., Idebenone, EPO (including biosimilars, mutants, and mimetics thereof), and/or antioxidants).

In order to test for thalassemia, a subject may be tested for genetic defects (e.g., deletions) in one or more genes encoding globin chains of the hemoglobin molecule, or deletions in the 16p chromosome. Examples of such genes include but are not limited to: HBA1, HBA2, or HBB. A subject testing positive for a genetic defect associated with thalassemia may be treated with one or more compounds described herein (e.g., compounds of Formula I, dimebolin, etc.). In some cases, the one or more compounds (e.g., compounds of Formula I, dimebolin, etc.) may be combined with one or more other compounds or treatments known to be effective against thalassemia (e.g., chronic blood transfusion therapy, iron chelation, splenectomy, allogeneic hematopoietic transplantation). In some cases, the one or more compounds may be combined with one or more other compounds or treatments known to be effective against thalassemia such as one or more of the following: iron chelators such as deferoxamine and deferasirox; antioxidants used to reduce preferryl-Hb such as indicaxanthin; drugs used to lower lung hypertension such as sildenafil; nifedine (a vasodilator which may also reduce iron overload); hydroxyurea; Gardos channel blockers such as seniapoc; drugs to modify hemoglobin switching including phytochemicals such as nicosan; folic acid; drugs used to treat vaso-occlusive crises including analgesics such as NSAIDS and opiods; and therapies used to treat acute chest crises, including oxygen supplementation for hypoxia, and antibiotics, such as quinolones or macrolides. In some cases, the one or more compounds (e.g., compounds of Formula I, dimebolin, etc.) may be combined with EPO (including biosimilars, mutants, and mimetics thereof) and/or antioxidants.

In order to test for sickle cell anemia or sickle cell disease, a subject may be tested for genetic defects in the β-gene (e.g., HbS). A subject testing positive for a genetic defect associated with sickle cell anemia may be treated with one or more compounds described herein (e.g., compounds of Formula I, dimebolin, etc.). In some cases, the one or more compounds may be combined with one or more other compounds or treatments known to be effective against sickle cell anemia or sickle cell disease (e.g., hydroxyuria, cyanate, folic acid). In some cases, the one or more compounds may be combined with one or more other compounds or treatments known to be effective against sickle cell disease or anemia such as one or more of the following: iron chelators such as deferoxamine and deferasirox; antioxidants used to reduce preferryl-Hb such as indicaxanthin; drugs used to lower lung hypertension such as sildenafil; nifedine (a vasodilator which may also reduce iron overload); hydroxyurea; Gardos channel blockers such as seniapoc; drugs to modify hemoglobin switching including phytochemicals such as nicosan; folic acid; drugs used to treat vaso-occlusive crises including analgesics such as NSAIDS and opiods; and therapies used to treat acute chest crises, including oxygen supplementation for hypoxia, and antibiotics, such as quinolones or macrolides. In some cases, the one or more compounds (e.g., compounds of Formula I, dimebolin, etc.) may be combined with EPO (including biosimilars, mutants, and mimetics thereof) and/or antioxidants.

### Use of Compounds in Research Applications, Experimental Systems, and Assays

The compounds of the invention can also be used in research applications. They can be used in vitro, in vivo, or ex vivo experiments to modulate one or more energy biomarkers in an experimental system. Such experimental systems can be cell samples, tissue samples, cell components or mixtures of cell components, partial organs, whole organs, or organisms. Any one or more of the compounds of formula I, can be used in experimental systems or research applications. Such research applications can include, but are not limited to, use as assay reagents, elucidation of biochemical pathways, or evaluation of the effects of other agents on the metabolic state of the experimental system in the presence/absence of one or more compounds of the invention.

Additionally, the compounds of the invention can be used in biochemical tests or assays. Such tests can include incubation of one or more compounds of the invention with a tissue or cell sample from a subject to evaluate a subject's potential response (or the response of a specific subset of subjects) to administration of said one or more compounds, or to determine which compound of the invention produces the optimum effect in a specific subject or subset of subjects. One such test or assay would involve 1) obtaining a cell sample or tissue sample from a subject in which modulation of one or more energy biomarkers can be assayed; 2) administering one or more compounds of the invention to the cell sample or tissue sample; and 3) determining the amount of modulation of the one or more energy biomarkers after administration of the one or more compounds, compared to the status of the energy biomarker prior to administration of the one or more compounds. Another such test or assay would involve 1) obtaining a cell sample or tissue sample from a subject in which modulation of one or more energy biomarkers can be assayed; 2) administering at least two compounds of the invention to the cell sample or tissue sample; 3) determining the amount of modulation of the one or more energy biomarkers after administration of the at least two compounds, compared to the status of the energy biomarker prior to administration of the at least compounds; and 4) selecting a compound for use in treatment, suppression, or modulation based on the amount of modulation determined in step 3).

### Screening Compounds for Effect on Modulating Energy Biomarkers

Compounds may be tested for their ability to modulate the level of one or more energy biomarkers described herein. The test compounds may be individual small molecules of choice. For example, the test compound may be a compound of Formula I. The test compounds may be compounds from a combinatorial library, i.e., a collection of diverse chemical compounds generated by either chemical synthesis or biological synthesis by combining a number of chemical “building blocks.” In some cases, the “building blocks” are individual chemical constituents capable of forming a compound of interest. For example, the “building blocks” may be a set of chemical constituents, each of which is capable of forming a compound of Formula I when combined with one or more other chemical constituents.

A sample may be combined with more than one compound from a combinatorial library. Methods of deconvoluting sample mixtures from a combinatorial library are well known in the art.

Compounds (including compounds having a structure other than that of Formula I) may be screened by the methods described herein, and can be synthesized through such combinatorial mixing of chemical building blocks. Preparation and screening of combinatorial chemical libraries are well known in the art. Combinatorial chemical libraries include, but are not limited to: diversomers such as hydantoins, benzodiazepines, and dipeptides, as described in, e.g., Hobbs et al., (1993), Proc. Natl Acad. Sci. U.S.A., 90:6909-6913; analogous organic syntheses of small compound libraries, as described in Chen et al., (1994), J. Amer. Chem. Soc., 116:2661-2662; and small organic molecule libraries containing, e.g., thiazolidinones and metathiazanones (U.S. Pat. No. 5,549,974), pyrrolidines (U.S. Pat. Nos. 5,525,735 and 5,519,134), benzodiazepines (U.S. Pat. No. 5,288,514). Additionally, numerous combinatorial libraries are commercially available from, e.g., ComGenex (Princeton, N.J.); Asinex (Moscow, Russia); Tripos, Inc. (St. Louis, Mo.); ChemStar, Ltd. (Moscow, Russia); 3D Pharmaceuticals (Exton, Pa.); and Martek Biosciences (Columbia, Md.).

Promising test compounds may be screened in secondary screens for toxicity or effectiveness. In some cases, a promising test agent may be tested along with a second compound, particularly a compound with a known therapeutic effect, in order to measure synergism between the two compounds.

Any energy biomarker described herein may be used as a read-out in the screen. For example, compounds may be screened for their ability to enhance or reduce the level of a biomarker described herein (e.g., lactic acid).

### Pharmaceutical Formulations

The compounds described herein can be formulated as pharmaceutical compositions by formulation with additives such as pharmaceutically acceptable excipients, pharmaceutically acceptable carriers, and pharmaceutically acceptable vehicles. Suitable pharmaceutically acceptable excipients, carriers and vehicles include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), and “Remington: The Science and Practice of Pharmacy,” Lippincott Williams & Wilkins, Philadelphia, 20th edition (2003) and 21st edition (2005), incorporated herein by reference.

A pharmaceutical composition can comprise a unit dose formulation, where the unit dose is a dose sufficient to have a therapeutic or suppressive effect or an amount effective to modulate, normalize, or enhance an energy biomarker. The unit dose may be sufficient as a single dose to have a therapeutic or suppressive effect or an amount effective to modulate, normalize, or enhance an energy biomarker. Alternatively, the unit dose may be a dose administered periodically in a course of treatment or suppression of a disorder, or to modulate, normalize, or enhance an energy biomarker.

Pharmaceutical compositions containing the compounds of the invention may be in any form suitable for the intended method of administration, including, for example, a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof.

Time-release or controlled release delivery systems may be used, such as a diffusion controlled matrix system or an erodible system, as described for example in: Lee, “Diffusion-Controlled Matrix Systems”, pp. 155-198 and Ron and Langer, “Erodible Systems”, pp. 199-224, in “Treatise on Controlled Drug Delivery”, A. Kydonieus Ed., Marcel Dekker, Inc., New York 1992. The matrix may be, for example, a biodegradable material that can degrade spontaneously in situ and in vivo for, example, by hydrolysis or enzymatic cleavage, e.g., by proteases. The delivery system may be, for example, a naturally occurring or synthetic polymer or copolymer, for example in the form of a hydrogel. Exemplary polymers with cleavable linkages include polyesters, polyorthoesters, polyanhydrides, polysaccharides, poly(phosphoesters), polyamides, polyurethanes, poly(imidocarbonates) and poly(phosphazenes).

The compounds of the invention may be administered enterally, orally, parenterally, sublingually, by inhalation (e.g. as mists or sprays), rectally, or topically in dosage unit formulations containing conventional nontoxic pharmaceutically acceptable carriers, adjuvants, and vehicles as desired. For example, suitable modes of administration include oral, subcutaneous, transdermal, transmucosal, iontophoretic, intravenous, intraarterial, intramuscular, intraperitoneal, intranasal (e.g. via nasal mucosa), intraocular, subdural, rectal, gastrointestinal, and the like, and directly to a specific or affected organ or tissue. For delivery to the central nervous system, spinal and epidural administration, or administration to cerebral ventricles, can be used. Topical administration may also involve the use of transdermal administration such as transdermal patches or iontophoresis devices. The term parenteral as used herein includes subcutaneous injections, intravenous, intramuscular, intrasternal injection, or infusion techniques. The compounds are mixed with pharmaceutically acceptable carriers, adjuvants, and vehicles appropriate for the desired route of administration. Oral administration is a preferred route of administration, and formulations suitable for oral administration are preferred formulations. The compounds described for use herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, and in other suitable forms. The compounds can also be administered in liposome formulations. The compounds can also be administered as prodrugs, where the prodrug undergoes transformation in the treated subject to a form which is therapeutically effective. Additional methods of administration are known in the art.

Injectable preparations, for example, sterile injectable aqueous or oleaginous suspensions, maybe formulated according to the known art using suitable dispersing or wetting agents and suspending agents. The sterile injectable preparation may also be a sterile injectable solution or suspension in a nontoxic parenterally acceptable diluent or solvent, for example, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The compounds of the present invention can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq (1976).

The invention also provides articles of manufacture and kits containing materials useful for treating or suppressing oxidative stress diseases affecting normal electron flow in the cells, such as mitochondrial diseases, impaired energy processing disorders, neurodegenerative disorders and diseases of aging. The invention also provides kits comprising any one or more of the compounds of formula I. In some embodiments, the kit of the invention comprises the container described above.

In other aspects, the kits may be used for any of the methods described herein, including, for example, to treat an individual with a mitochondrial disorder, or to suppress a mitochondrial disorder in an individual.

The amount of active ingredient that may be combined with the carrier materials to produce a single dosage form will vary depending upon the host to which the active ingredient is administered and the particular mode of administration. It will be understood, however, that the specific dose level for any particular patient will depend upon a variety of factors including the activity of the specific compound employed, the age, body weight, body area, body mass index (BMI), general health, sex, diet, time of administration, route of administration, rate of excretion, drug combination, and the type, progression, and severity of the particular disease undergoing therapy. The pharmaceutical unit dosage chosen is usually fabricated and administered to provide a defined final concentration of drug in the blood, tissues, organs, or other targeted region of the body. The therapeutically effective amount or effective amount for a given situation can be readily determined by routine experimentation and is within the skill and judgment of the ordinary clinician.

Examples of dosages which can be used are an effective amount within the dosage range of about 0.1 mg/kg to about 300 mg/kg body weight, or within about 1.0 mg/kg to about 100 mg/kg body weight, or within about 1.0 mg/kg to about 50 mg/kg body weight, or within about 1.0 mg/kg to about 30 mg/kg body weight, or within about 1.0 mg/kg to about 10 mg/kg body weight, or within about 10 mg/kg to about 100 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight. Examples of effective amounts that may be administered to a subject include: about 1 mg, about 5 mg, 7.5 mg. about 10 mg, about 15 mg, about 20 mg, about 25 mg, about 30 mg, about 40 mg, about 50 mg, about 75 mg, or about 100 mg.

Compounds of the present invention may be administered in a single daily dose, or the total daily dosage may be administered in divided dosage of two, three or four times daily.

Administration of the compounds of the invention may be performed for periods of less than about a week, less than about a month, or less than about a year. In some embodiments, administration is performed for longer than one year.

### Combination Therapy

While the compounds of the invention can be administered as the sole active pharmaceutical agent, they can also be used in combination with one or more other therapeutic agents used in the treatment or suppression of disorders. The compounds of the invention are administered concomitantly, in the same composition, prior to or subsequent to administration of the one or more other therapeutic agents. This disclosure also provides formulations comprising combinations of one or more compounds of Formula I (e.g., dimebolin) and one or more additional therapeutic agents (e.g., erythropoietin (EPO), including mutants, biosimilars, or mimetics thereof, Coenzyme Q, vitamin E, Idebenone, MitoQ, vitamins, antioxidant compounds).

Representative agents useful in combination with the compounds of the invention for the treatment or suppression of mitochondrial diseases include, but are not limited to erythropoietin (EPO, including mutants, biosimilars, or mimetics thereof), Coenzyme Q, vitamin E, Idebenone, MitoQ, vitamins, and antioxidant compounds.

Erythropoietin (EPO) is a glycoprotein hormone that regulates growth and survival of erythroid progenitors. These erythroid progenitors mature into red blood cells. The native hormone is a mixture of isoforms. A number of recombinant rHuEPO (EPO biosimilars) products are available for use, including Epoetin alfa, Epoetin beta, Epoetin delta, and darbaepoetin (which is PEGylated), all of which have differing glycan structures. EPO mutant proteins useful in the methods of the invention include but are not limited to Synthetic Erythropoiesis Protein (SEP, Gryphon Therapeutics), and Continuous Erythropoietin Receptor Activator (CERA, Roche). EPO fusion proteins, and dimerized protein/peptide segments are also available for use in the methods of the invention. Additionally, EPO mimetics, including but not limited to EMP1 (EPO mimetic peptide 1, GGTYSCHFGPLTWVCKPQGG (a cyclic disulfide bridged peptide)), ERB1-7 (DREGCRRGWVGQCKAWFN, a cyclic disulfide bridged peptide), ERP (QRVEILEGRTECVKSNLRGRTRY, a linear peptide), Hematide™ (a dimeric peptide mimetic having a sequence unrelated to EPO), and CNTO-528 or CNTO-530 (EPO mimetic antibody fusion proteins produced using Centocor's technology Mimetibody™ having no sequence homology with EPO but acting as a erythropoietin receptor agonist) are useful in the methods of the invention. Small molecule mimetics of EPO are also useful in the methods of the invention, due to the ease of oral administration.

Antioxidant compounds include but are not limited to: ascorbic acid (vitamin C) and its salts, ascorbyl esters of fatty acids, ascorbic acid derivatives (e.g., magnesium ascorbyl phosphate, sodium ascorbyl phosphate, and ascorbyl sorbate), tocopherol (vitamin E), tocopherol sorbate, tocopherol acetate, other esters of tocopherol, retinoids (e.g. retinoic acid, retinol), carotenoids (e.g. .alpha.- and .beta.-carotene), xanthophylls (e.g. lutein, zeaxanthin), indicaxanthin, butylated hydroxy benzoic acids and their salts, 6-hydroxy-2,5,7,8-tetramethylchroman-2-carboxylic acid (commercially available under the tradename Trolox.™.), gallic acid and its alkyl esters, especially propyl gallate, uric acid and its salts and alkyl esters, sorbic acid and its salts, lipoic acid, amines (e.g., N,N-diethylhydroxylamine and amino-guanidine), sulfhydryl compounds (e.g., glutathione), dihydroxy fumaric acid and it salts, glycine pidolate, arginine pidolate, nordihydroguaiaretic acid, bioflavinoids, curcumin, lyseine, cysteine, methionine, citric acid, ethylenediamine tetraacetic acid (EDTA), sorbitol, tartaric acid, and phosphoric acid.

Representative therapeutic agents useful in combination with the compounds of the invention for the treatment of haemoglobinopathies such as thalassemia and sickle cell disease include but are not limited to: iron chelators such as deferoxamine and deferasirox; antioxidants used to reduce preferryl-Hb such as indicaxanthin; drugs used to lower lung hypertension such as sildenafil; nifedine (a vasodilator which may also reduce iron overload); hydroxyurea; Gardos channel blockers such as seniapoc; drugs to modify hemoglobin switching including phytochemicals such as nicosan; folic acid; drugs used to treat vaso-occlusive crises including analgesics such as NSAIDS and opioids; and therapies used to treat acute chest crises, including oxygen supplementation for hypoxia, and antibiotics, such as quinolones or macrolides.

When additional active agents are used in combination with the compounds of the present invention, the additional active agents may generally be employed in therapeutic amounts as indicated in the Physicians' Desk Reference (PDR) 53rd Edition (1999), which is incorporated herein by reference, or such therapeutically useful amounts as would be known to one of ordinary skill in the art.

The compounds of the invention and the other therapeutically active agents can be administered at the recommended maximum clinical dosage or at lower doses. Dosage levels of the active compounds in the compositions of the invention may be varied so as to obtain a desired therapeutic response depending on the route of administration, severity of the disease and the response of the patient. When administered in combination with other therapeutic agents, the therapeutic agents can be formulated as separate compositions that are given at the same time or different times, or the therapeutic agents can be given as a single composition.

### EXAMPLES

The invention will be further understood by the following nonlimiting examples.

### Biological Examples

### Example A

Screening Compounds of the Invention in Human Dermal Fibroblasts from Friedreich 's Ataxia Patients

An initial screen was performed to identify compounds effective for the amelioration of redox disorders. Test samples, 4 reference compounds (Idebenone, decylubiquinone, Trolox and α-tocopherol acetate), and solvent controls were tested for their ability to rescue FRDA fibroblasts stressed by addition of L-buthionine-(S,R)-sulfoximine (BSO), as described in Jauslin et al., Hum. Mol. Genet. 11(24):3055 (2002), Jauslin et al., FASEB J. 17:1972-4 (2003), and International Patent Application WO 2004/003565. Human dermal fibroblasts from Friedreich's Ataxia patients have been shown to be hypersensitive to inhibition of the de novo synthesis of glutathione (GSH) with L-buthionine-(S,R)-sulfoximine (BSO), a specific inhibitor of GSH synthetase (Jauslin et al., Hum. Mol. Genet. 11(24):3055 (2002)). This specific BSO-mediated cell death can be prevented by administration of antioxidants or molecules involved in the antioxidant pathway, such as α-tocopherol, selenium, or small molecule glutathione peroxidase mimetics. However, antioxidants differ in their potency, i.e. the concentration at which they are able to rescue BSO-stressed FRDA fibroblasts.

MEM (a medium enriched in amino acids and vitamins, catalog no. 1-31F24-I) and Medium 199 (M199, catalog no. 1-21F22-I) with Earle's Balanced Salts, without phenol red, were purchased from Bioconcept. Fetal Calf Serum was obtained from PAA Laboratories. Basic fibroblast growth factor and epidermal growth factor were purchased from PeproTech. Penicillin-streptomycin-glutamine mix, L-buthionine (S,R)-sulfoximine, (+)-α-tocopherol acetate, decylubiquinone, and insulin from bovine pancreas were purchased from Sigma. Trolox (6-hydroxy-2,5,7,8-tetramethylchromane-2-carboxylic acid) was obtained from Fluka. Idebenone was obtained from Chemo Iberica. Calcein AM was purchased from Molecular Probes. Cell culture medium was made by combining 125 ml M199 EBS, 50 ml Fetal Calf Serum, 100 U/ml penicillin, 100 μg/ml streptomycin, 2 mM glutamine, 10 μg/ml insulin, 10 ng/ml EGF, and 10 ng/ml bFGF; MEM EBS was added to make the volume up to 500 ml. A 10 mM BSO solution was prepared by dissolving 444 mg BSO in 200 ml of medium with subsequent filter-sterilization. During the course of the experiments, this solution was stored at +4° C. The cells were obtained from the Coriell Cell Repositories (Camden, N.J.; repository number GM04078) and grown in 10 cm tissue culture plates. Every third day, they were split at a 1:3 ratio.

The test samples were supplied in 1.5 ml glass vials. The compounds were diluted with DMSO, ethanol or PBS to result in a 5 mM stock solution. Once dissolved, they were stored at −20° C. Reference antioxidants (Idebenone, decylubiquinone, α-tocopherol acetate and trolox) were dissolved in DMSO.

Test samples were screened according to the following protocol:
A culture with FRDA fibroblasts was started from a 1 ml vial with approximately 500,000 cells stored in liquid nitrogen. Cells were propagated in 10 cm cell culture dishes by splitting every third day in a ratio of 1:3 until nine plates were available. Once confluent, fibroblasts were harvested. For 54 micro titer plates (96 well-MTP) a total of 14.3 million cells (passage eight) were re-suspended in 480 ml medium, corresponding to 100 μl medium with 3,000 cells/well. The remaining cells were distributed in 10 cm cell culture plates (500,000 cells/plate) for propagation. The plates were incubated overnight at 37° C. in an atmosphere with 95% humidity and 5% CO₂ to allow attachment of the cells to the culture plate.

MTP medium (243 μl) was added to a well of the microtiter plate. The test compounds were unfrozen, and 7.5 μl of a 5 mM stock solution was dissolved in the well containing 243 μl medium, resulting in a 150 μM master solution. Serial dilutions from the master solution were made. The period between the single dilution steps was kept as short as possible (generally less than 1 second).

Plates were kept overnight in the cell culture incubator. The next day, 10 μl of a 10 mM BSO solution were added to the wells, resulting in a 1 mM final BSO concentration. Forty-eight hours later, three plates were examined under a phase-contrast microscope to verify that the cells in the 0% control (wells E1-H1) were clearly dead. The medium from all plates was discarded, and the remaining liquid was removed by gently tapping the plate inversed onto a paper towel.

100 μl of PBS containing 1.2 μM Calcein AM were then added to each well. The plates were incubated for 50-70 minutes at room temperature. After that time the PBS was discarded, the plate gently tapped on a paper towel and fluorescence (excitation/emission wavelengths of 485 nm and 525 nm, respectively) was read on a Gemini fluorescence reader. Data was imported into Microsoft Excel (EXCEL is a registered trademark of Microsoft Corporation for a spreadsheet program) and used to calculate the EC₅₀ concentration for each compound.

The compounds were tested three times, i.e., the experiment was performed three times, the passage number of the cells increasing by one with every repetition.

The solvents (DMSO, ethanol, PBS) had neither a detrimental effect on the viability of non-BSO treated cells nor did they have a beneficial influence on BSO-treated fibroblasts even at the highest concentration tested (1%). None of the compounds showed auto-fluorescence. The viability of non-BSO treated fibroblasts was set as 100%, and the viability of the BSO- and compound-treated cells was calculated as relative to this value.

The following table summarizes the EC₅₀ for the four control compounds.

| | EC₅₀ [μM] | | | | |
|---|---|---|---|---|---|
| Compound | Value 1 | Value 2 | Value 3 | Average | Stdev |
| Decylubiquinone | 0.05 | 0.035 | 0.03 | 0.038 | 0.010 |
| alpha-Tocopherol acetate | 0.4 | 0.15 | 0.35 | 0.30 | 0.13 |
| Idebenone | 1.5 | 1 | 1 | 1.2 | 0.3 |
| Trolox | 9 | 9 | 8 | 8.7 | 0.6 |

The following compounds were tested by this method and exhibited protection against Friedreich's ataxia with an EC₅₀ as follows:
2,8-dimethyl-5-(2-(6-methylpyridin-3-yl)ethyl)-2,3,4,5-tetrahydro-1H-pyrido[4,3-b]indole (dimebolin): 1.9 μM.
5-benzyl-2-methyl-2,3,4,5-tetrahydro-1H-pyrido[4,3-b]indole: 4.26 μM.
8-methyl-1,3,4,4a,5,9b-tetrahydro-1H-pyrido[4,3-b]indole: 4.1 μM.

Compounds tested by this method are considered to be active if they exhibit protection against Friedreich's ataxia with an EC₅₀ of less than about 150 nM, about 500 nM, about 1.0 μM, about 1.5 μM, about 2.0 μM, about 2.5 μM, about 3.0 μM, about 3.5 μM, about 4.0 μM, about 4.5 μM , or about 5.0 μM.

### Example B

Screening Compounds of the Invention in Fibroblasts from Leber 's Hereditary Optic Neuropathy Patients

Compounds of the invention were screened as described in Example A, but substituting FRDA cells with Leber's Hereditary Optic Neuropathy (LHON) cells obtained from the Coriell Cell Repositories (Camden, N.J.; repository number GM03858). The compounds were tested for their ability to rescue human dermal fibroblasts from LHON patients from oxidative stress.

The following compounds were tested by this method and exhibited protection against LHON with an EC₅₀ as follows:
5-benzyl-2-methyl-2,3,4,5-tetrahydro-1H-pyrido[4,3-b]indole: 1.2 μM.

Compounds tested by this method are considered to be active if they exhibit protection against Leber's Hereditary Optic Neuropathy with an EC₅₀ of less than about 150 nM, about 500 nM, about 1.0 μM, about 1.5 μM, about 2.0 μM, about 2.5 μM, about 3.0 μM, about 3.5 μM, about 4.0 μM, about 4.5 μM, or about 5.0 μM.

### Example C

Screening Compounds of the Invention in Fibroblasts from CoQ10 Deficient Patients

Compounds of the invention were tested using a screen similar to the one described in Example A, but substituting FRDA cells with cells obtained from CoQ10 deficient patients harboring a CoQ2 mutation. The compounds were tested for their ability to rescue human dermal fibroblasts from CoQ10 deficient patients from oxidative stress.

The following compounds were tested by this method and exhibited protection against CoQ10 with an EC₅₀ as follows:
2,8-dimethyl-5-(2-(6-methylpyridin-3-yl)ethyl)-2,3,4,5-tetrahydro-1H-pyrido[4,3-b]indole (dimebolin): 0.23 μM.
5-benzyl-2-methyl-2,3,4,5-tetrahydro-1H-pyrido[4,3-b]indole: 3.35 μM.
8-methyl-1,3,4,4a,5,9b-tetrahydro-1H-pyrido[4,3-b]indole: 7 μM.

Compounds tested by this method are considered to be active if they exhibit protection against CoQ10 deficiency with an EC₅₀ of less than about 150 nM, about 500 nM, about 1.0 μM, about 1.5 μM, about 2.0 μM, about 2.5 μM, about 3.0 μM, about 3.5 μM, about 4.0 μM, about 4.5 μM, or about 5.0 μM.

### Example D

Screening Compounds of the Invention in Fibroblasts from Parkinson 's Disease Patients

Compounds of the invention were tested using a screen similar to the one described in Example A described in Example A, but substituting FRDA cells with Parkinson's Disease (PD) cells obtained from the Coriell Cell Repositories (Camden, N.J.; repository number AG20439). The compounds were tested for their ability to rescue human dermal fibroblasts from Parkinson's Disease patients from oxidative stress.

The following compounds were tested by this method and exhibited protection against Parkinson's with an EC₅₀ as follows.
5-benzyl-2-methyl-2,3,4,5-tetrahydro-1H-pyrido[4,3-b]indole: 0.93 μM.
8-methyl-1,3,4,4a,5,9b-tetrahydro-1H-pyrido[4,3-b]indole: 3.68 μM.

Compounds tested by this method are considered to be active if they exhibit protection against Parkinson's disease with an EC₅₀ of less than about 150 nM, about 500 nM, about 1.0 μM, about 1.5 μM, about 2.0 μM, about 2.5 μM, about 3.0 μM, about 3.5 μM, about 4.0 μM about 4.5 μM or about 5.0 μM.

### Example E

### Mitochondrial Myopathy, Encephalopathy, Lactacidosis, Stroke (MELAS) (Prophetic Example)

Subjects are screened either by genetic testing or by questionnaire for a genetic defect associated with MELAS (e.g., A3243G or A8344G mitochondrial mutation) present either in their own genome or in the mitochondrial genome of a maternal relative. Subjects testing positive for one or more genetic defects (or with a maternal relative carrying such defects) are optionally tested for the level of one or more energy biomarkers such as lactic acid (lactate) levels ; pyruvic acid (pyruvate) levels ; lactate/pyruvate ratios; phosphocreatine levels; NADH (NADH+H⁺) levels; NADPH (NADPH+H⁺) levels; NAD levels; NADP levels; ATP levels; reduced coenzyme Q (CoQ^{red}) levels; oxidized coenzyme Q (CoQ^{ox}) levels; total coenzyme Q (CoQ^{tot}) levels; oxidized cytochrome C levels; reduced cytochrome C levels; oxidized cytochrome C/reduced cytochrome C ratio; acetoacetate levels; β-hydroxy butyrate levels; acetoacetate/β-hydroxy butyrate ratio, 8-hydroxy-2′-deoxyguanosine (8-OHdG) levels; levels of reactive oxygen species; levels of oxygen consumption (VO₂); or levels of carbon dioxide output (VCO₂). Optionally, subjects are tested for energy biomarkers by the following measures: respiratory quotient (VCO₂/VO₂); exercise tolerance; or anaerobic threshold. Subjects testing positive for a genetic defect (or with a maternal relative carrying such defect) but negative for an abnormal energy biomarker (or level thereof) are treated with a compound of Formula I in a dosage suitable to exert a prophylactic effect. Subjects testing positive for one or more genetic defects (or with a maternal relative carrying such defects) and positive for an abnormal level of energy biomarker are treated with an effective amount of a compound of Formula I. Optionally, one or more energy biomarkers are monitored over time. Optionally, the compound of Formula I is combined with a drug or therapy known to be effective against MELAS.

### Example F

Myoclonic Epilepsy with Ragged Red Fibers (MERRF) (Prophetic Example)

Subjects are screened either by genetic testing or by questionnaire for a genetic defect associated with MERRF present either in their own genome or in the mitochondrial genome of a maternal relative. Subjects testing positive for one or more genetic defects (or with a maternal relative carrying such defects) are optionally tested for energy biomarkers as described in Example E. Subjects testing positive for a genetic defect (or with a maternal relative carrying such defect) but negative for an abnormal energy biomarker are treated with a compound of Formula I in a dosage suitable to exert a prophylactic effect. Subjects testing positive for one or more genetic defects (or with a maternal relative carrying such defects) and positive for an abnormal level of energy biomarker are treated with an effective amount of a compound of Formula I. Optionally, one or more energy biomarkers are monitored over time. Optionally, the compound of Formula I is combined with a drug or therapy known to be effective against MERRF.

### Example G

### Friedreich 's Ataxia (FRDA) (Prophetic Example)

Subjects are screened either by genetic testing or by questionnaire for a genetic defect associated with FRDA present either in their own genome or in the mitochondrial genome of a maternal relative. Subjects testing positive for one or more genetic defects (or with a maternal relative carrying such defects) are optionally tested for energy biomarkers as described in Example E. Subjects testing positive for a genetic defect (or with a maternal relative carrying such defect) but negative for an abnormal energy biomarker are treated with a compound of Formula I in a dosage suitable to exert a prophylactic effect. Subjects testing positive for one or more genetic defects (or with a maternal relative carrying such defects) and positive for an abnormal level of energy biomarker are treated with an effective amount of a compound of Formula I. Optionally, one or more energy biomarkers are monitored over time. Optionally, the compound of Formula I is combined with a drug or therapy known to be effective against FRDA.

### Example H

### Co-Enzyme Q10 (CoQ10) Deficiency (Prophetic Example)

Subjects are screened either by genetic testing or by questionnaire for a genetic defect associated with CoQ10 deficiency present either in their own genome or in the mitochondrial genome of a maternal relative. Subjects testing positive for one or more genetic defects (or with a maternal relative carrying such defects) are optionally tested for energy biomarkers as described in Example E. Subjects testing positive for a genetic defect (or with a maternal relative carrying such defect) but negative for an abnormal energy biomarker are treated with a compound of Formula I in a dosage suitable to exert a prophylactic effect. Subjects testing positive for one or more genetic defects (or with a maternal relative carrying such defects) and positive for an abnormal level of energy biomarker are treated with an effective amount of a compound of Formula I. Optionally, one or more energy biomarkers are monitored over time. Optionally, the compound of Formula I is combined with a drug or therapy known to be effective against CoQ10 deficiency.

### Example I

### Leigh Disease or Leigh Syndrome, Kearns-Sayre Syndrome (KSS) (Prophetic Example)

Subjects are screened either by genetic testing or by questionnaire for a genetic defect associated with Leigh Disease, Leigh Syndrome or KSS either in their own genome or in the mitochondrial genome of a maternal relative. Subjects testing positive for one or more genetic defects (or with a maternal relative carrying such defects) are optionally tested for energy biomarkers as described in Example E. Subjects testing positive for a genetic defect (or with a maternal relative carrying such defect) but negative for an abnormal energy biomarker are treated with a compound of Formula I in a dosage suitable to exert a prophylactic effect. Subjects testing positive for one or more genetic defects (or with a maternal relative carrying such defects) and positive for an abnormal level of energy biomarker are treated with an effective amount of a compound of Formula I. Optionally, one or more energy biomarkers are monitored over time. Optionally, the compound of Formula I is combined with a drug or therapy known to be effective against Leigh Disease, Leigh Syndrome or KSS.

### Example J

### Human Trials (Prophetic Example)

At enrollment, subjects must have a diagnosis of MELAS, MERRF, FRDA, CoQ10 deficiency, Leigh Disease, Leigh Syndrome, or KSS, or of an abnormal energy biomarker. Subjects with MELAS, MERRF, FRDA, CoQ10 deficiency, Leigh Disease, Leigh Syndrome, KSS, or abnormal energy biomarker are treated with a compound of Formula I (e.g., dimebolin) for up to 3 years. Each group of test subjects is treated QD, BID or TID with different dose strengths of compound of Formula I. The drug is self administered by each subject orally once, twice, or three times a day. The dose strengths include placebo (0 mg) and increasing dosages. To enhance patient compliance, compound of Formula I can be administered as a slow release formulation.

Patients are supplied with drug (or placebo) and required to record the administration of each drug dose in a diary. Patients are assessed at the start of the study and every 2 months for the duration of the study for energy biomarkers as described in Example E. Each patient exam will include assessments of safety, energy biomarker level, and/or other symptom of their disease.

Although the foregoing invention has been described in some detail by way of illustration and example for purposes of clarity of understanding, it is apparent to those skilled in the art that certain minor changes and modifications will be practiced. It will likewise be apparent to skilled artisans that such embodiments are provided by way of example only. Therefore, the description and examples should not be construed as limiting the scope of the invention. Numerous variations, changes, and substitutions will occur to those skilled in the art without departing from the invention. It is intended that the following claims define the scope of the invention and that methods and structures within the scope of these claims and their equivalents be covered thereby.

What is claimed is:

1. A method of treating a subject having an oxidative stress disorder, or at risk for having an oxidative stress disorder, comprising administering to the subject a therapeutically effective amount of one or more compounds of Formula I: 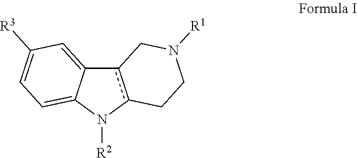
or its pharmaceutically acceptable salts, prodrugs, solvates, or hydrates thereof;
wherein R¹ is hydrogen, alkyl, aralkyl or heteroaralkyl;
R² is hydrogen, aralkyl, or heteroaralkyl;
R³ is hydrogen, alkyl, or halo;
a bond represented by a solid line accompanied by a dotted line is a single or a double bond; wherein the oxidative stress disorder is a haemoglobinopathy or caused by a defect in a gene encoding a mitochondrial protein or tRNA; and
wherein the oxidative stress disorder is not Leber's Hereditary Optic Neuropathy (LHON)

2. The method of claim 1 wherein when R¹ and/or R³ is alkyl, the R¹ and/or R³ is methyl.

3. The method of claim 1 wherein when R¹ and/or R² is an aralkyl moiety, the aryl of the aralkyl moiety is phenyl and the alkyl of the aralkyl moiety is methyl.

4. The method of claim 1 wherein when R¹ and/or R² is a heteroaralkyl moiety, the heteroaryl of the heteroaralkyl moiety is pyridinyl.

5. The method of claim 1 wherein the compound of Formula I has a structure wherein, R¹ is hydrogen, C₁-C₄-alkyl, benzyl or 3-(pyridin-3-yl)propyl;
R² is hydrogen, benzyl or 2-(6-methylpyridin-3-yl)ethyl);
R³ is hydrogen, C₁-C₄-alkyl, or halogen.

6. The method of claim 5 wherein R¹ is C₁-C₄-alkyl, benzyl or 3-(pyridin-3-yl)propyl.

7. The method of claim 6 wherein when R¹ and/or R³ is C₁-C₄-alkyl, the C₁-C₄-alkyl is unsubstituted.

8. The method of claim 5, wherein the compound is selected from the group consisting of dimebolin (2,8-dimethyl-5-(2-(6-methylpyridin-3-yl)ethyl)-2,3,4,5-tetrahydro-1H-pyrido[4,3-b]indole); 8-chloro-2-methyl-5-(2-(6-methylpyridin-3-yl)ethyl)-2,3,4,5-tetrahydro-1H-pyrido[4,3-b]indole;mebhydroline (5-benzyl-2-methyl-2,3,4,5-tetrahydro-1H-pyrido[4,3-b]indole); 2,8-dimethyl-1,3,4,4a,5,9b-hexahydro-1H-pyrido[4,3-b]indole; 8-fluoro-2-(3-(pyridin-3-yl)propyl)-2,3,4,5-tetrahydro-1H-pyrido[4,3-b]indole; and 8-methyl-1,3,4,4a,5,9b-tetrahydro-1H-pyrido[4,3-b]indole.

9. The method of claim 8 wherein the compound is a hydrochloride, sulfate, phosphate, fumarate, maleate, palmitate, tosylate, mesylate, acetate, or citrate salt.

10. The method of claim 5, wherein the compound is dimebolin (2,8-dimethyl-5-(2-(6-methylpyridin-3-yl)ethyl)-2,3,4,5-tetrahydro-1H-pyrido[4,3-b]indole).

11. The method of claim 1 wherein said administering further comprises administering the one or more compounds of Formula I with a pharmaceutically acceptable excipient.

12. The method of claim 1 wherein the oxidative stress disorder is caused by a defect in a gene encoding a mitochondrial protein or tRNA.

13. The method of claim 12, wherein the defect results in a respiratory chain disorder.

14. The method of claim 12, wherein the defect causes a disorder selected from the group consisting of Myoclonic Epilepsy with Ragged Red Fibers (MERRF); Mitochondrial Myopathy, Encephalopathy, Lactacidosis, Stroke (MELAS); chronic progressive external ophthalmoplegia (CPEO); Leigh Disease; Kearns-Sayre Syndrome (KSS); Friedreich's Ataxia (FRDA); Co-Enzyme Q10 (CoQ10) Deficiency; Complex I Deficiency; Complex II Deficiency; Complex III Deficiency; Complex IV Deficiency; and Complex V Deficiency.

15. The method of claim 1 wherein the oxidative stress disorder is a haemoglobinopathy.

16. The method of claim 15, wherein the haemoglobinopathy is thalassemia or sickle-cell disease.

17. A method of modulating the level of energy biomarkers in a subject comprising administering to the subject an effective amount of one or more compounds wherein the one or more compounds normalizes one or more energy markers in a subject or enhances or reduces the level of each of one or more energy biomarkers in the subject by more than 10%.

18. The method of claim 17, wherein the one or more compounds are one or more compounds of Formula I: 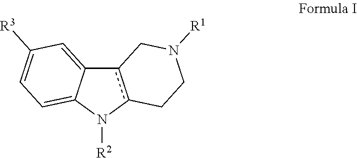
or its pharmaceutically acceptable salts, prodrugs, solvates, or hydrates thereof;
wherein R¹ is hydrogen, alkyl, aralkyl or heteroaralkyl;
R² is hydrogen, aralkyl, or heteroaralkyl;
R³ is hydrogen, alkyl, or halo; and
the bond represented by a solid line accompanied by a dotted line is a single or a double bond.

19. The method of claim 18, wherein the one or more compounds are one or more compounds of Formula I: 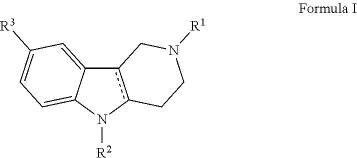
or its pharmaceutically acceptable salts, prodrugs, solvates, or hydrates thereof, wherein,
R¹ is hydrogen, C₁-C₄-alkyl, benzyl or 3-(pyridin-3-yl)propyl;
R² is hydrogen, benzyl or 2-(6-methylpyridin-3-yl)ethyl);
R³ is hydrogen, C₁-C₄-alkyl, or halogen; and
the bond represented by a solid line accompanied by a dotted line is a single or double bond.

20. The method of claim 19 wherein R¹ is C₁-C₄-alkyl, benzyl or 3-(pyridin-3-yl)propyl.

21. The method of claim 20 wherein when R¹ and/or R³ is C₁-C₄-alkyl, the C₁-C₄-alkyl is unsubstituted.

22. The method of claim 19, wherein the compound is selected from the group consisting of dimebolin (2,8-dimethyl-5-(2-(6-methylpyridin-3-yl)ethyl)-2,3,4,5-tetrahydro-1H-pyrido[4,3-b]indole), 8-chloro-2-methyl-5-(2-(6-methylpyridin-3-yl)ethyl)-2,3,4,5-tetrahydro-1H-pyrido[4,3-b]indole, mebhydroline (5-benzyl-2-methyl-2,3,4,5-tetrahydro-1H-pyrido[4,3-b]indole), 2,8-dimethyl-1,3,4,4a,5,9b-hexahydro-1H-pyrido[4,3-b]indole, 8-fluoro-2-(3-(pyridin-3-yl)propyl)-2,3,4,5-tetrahydro-1H-pyrido[4,3-b]indole, and 8-methyl-1,3,4,4a,5,9b-tetrahydro-1H-pyrido[4,3-b]indole.

23. The method of claim 19, wherein the compound is dimebolin (2,8-dimethyl-5-(2-(6-methylpyridin-3-yl)ethyl)-2,3,4,5-tetrahydro-1H-pyrido[4,3-b]indole).

24. The method of claim 18, further comprising administering the one or more compounds of Formula I with a pharmaceutically acceptable excipient.

25. The method of claim 18, further comprising measuring the level of one or more energy biomarkers in the subject prior to or following the administering of the compound.

26. The method of claim 18, where the one or more energy biomarkers is selected from the group consisting of: lactic acid (lactate) levels; pyruvic acid (pyruvate) levels; lactate/pyruvate ratios; phosphocreatine levels; NADH (NADH+H⁺) levels; NADPH (NADPH+H⁺) levels; NAD levels; NADP levels; ATP levels; reduced coenzyme Q (CoQ^{red}) levels; oxidized coenzyme Q (CoQ^{ox}) levels; total coenzyme Q (CoQ^{tot}) levels; oxidized cytochrome C levels; reduced cytochrome C levels; oxidized cytochrome C/reduced cytochrome C ratio; acetoacetate levels; β-hydroxy butyrate levels; acetoacetate/β-hydroxy butyrate ratio, 8-hydroxy-2′-deoxyguanosine (8-OHdG) levels; levels of reactive oxygen species; levels of oxygen consumption (VO₂) and levels of carbon dioxide output (VCO₂).

27. The method of claim 18, wherein the subject has an abnormal level of one or more energy biomarkers.

28. The method of claim 18, wherein the subject has a normal level of one or more energy biomarkers.

29. The method of claim 18, wherein the subject has an abnormal respiratory quotient (VCO2/VO2), an abnormal result from an exercise tolerance test, or an abnormal anaerobic threshold.

30. A method of treating a subject having an oxidative stress disorder comprising:
(a) testing the subject for a genetic defect; and
(b) administering to the subject a therapeutically effective amount of one or more compounds of Formula I: 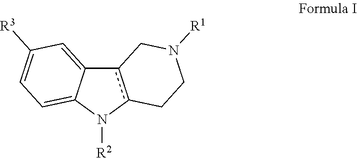
or its pharmaceutically acceptable salts, prodrugs, solvates, or hydrates thereof;
wherein R¹ is hydrogen, alkyl, aralkyl or heteroaralkyl;
R² is hydrogen, aralkyl, or heteroaralkyl;
R³ is hydrogen, alkyl, or halo; and
a bond represented by a solid line accompanied by a dotted line is a single or a double bond.

31. The method of claim 30, wherein the compound of Formula I is the compound wherein;
R¹ is hydrogen, C₁-C₄-alkyl, benzyl or 3-(pyridin-3-yl)propyl;
R² is hydrogen, benzyl or 2-(6-methylpyridin-3-yl)ethyl); and
R³ is hydrogen, C₁-C₄-alkyl, or halogen.

32. The method of claim 31, wherein R¹ is C₁-C₄-alkyl, benzyl or 3-(pyridin-3-yl)propyl.

33. The method of claim 32 wherein when R¹ and/or R³ is C₁-C₄-alkyl, the C₁-C₄-alkyl is unsubstituted.

34. The method of claim 31, wherein the compound is selected from the group consisting of dimebolin (2,8-dimethyl-5-(2-(6-methylpyridin-3-yl)ethyl)-2,3,4,5-tetrahydro-1H-pyrido[4,3-b]indole); 8-chloro-2-methyl-5-(2-(6-methylpyridin-3-yl)ethyl)-2,3,4,5-tetrahydro-1H-pyrido[4,3-b]indole; mebhydroline (5-benzyl-2-methyl-2,3,4,5-tetrahydro-1H-pyrido[4,3-b]indole); 2,8-dimethyl-1,3,4,4a,5,9b-hexahydro-1H-pyrido[4,3-b]indole, 8-fluoro-2-(3-(pyridin-3-yl)propyl)-2,3,4,5-tetrahydro-1H-pyrido[4,3-b]indole; and 8-methyl-1,3,4,4a,5,9b-tetrahydro-1H-pyrido[4,3-b]indole.

35. The method of claim 31, wherein the compound is dimebolin (2,8-dimethyl-5-(2-(6-methylpyridin-3-yl)ethyl)-2,3,4,5-tetrahydro-1H-pyrido[4,3-b]indole).

36. The method of claim 30, further comprising administering the one or more compounds of Formula I with a pharmaceutically acceptable excipient.

37. The method of claim 30, wherein the genetic defect is a defect in a gene encoding a mitochondrial protein or tRNA.

38. The method of claim 37, wherein the genetic defect results in a respiratory chain disorder.

39. The method of claim 37, where the genetic defect causes a disorder selected from the group consisting of Myoclonic Epilepsy with Ragged Red Fibers (MERRF); Mitochondrial Myopathy, Encephalopathy, Lactacidosis, Stroke (MELAS); chronic progressive external ophthalmoplegia (CPEO); Leigh Disease; Kearns-Sayre Syndrome (KSS); Friedreich's Ataxia (FRDA); Co-Enzyme Q10 (CoQ10) Deficiency; Complex I Deficiency; Complex II Deficiency; Complex III Deficiency; Complex IV Deficiency; and Complex V Deficiency.

40. The method of claim 30, wherein the genetic defect causes haemoglobinopathy.

41. The method of claim 40, wherein the haemoglobinopathy is thalassemia or sickle-cell disease.

42. A formulation comprising a first and second compound wherein the first compound is effective against an oxidative stress disorder and the second compound is one or more compounds of Formula I: 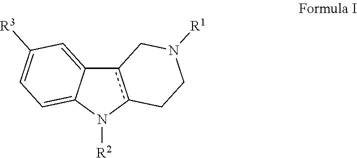
or its pharmaceutically acceptable salts, prodrugs, solvates, or hydrates thereof;
wherein R¹ is hydrogen, alkyl, aralkyl or heteroaralkyl;
R² is hydrogen, aralkyl, or heteroaralkyl;
R³ is hydrogen, alkyl, or halo;
a bond represented by a solid line accompanied by a dotted line is a single or a double bond.

43. The formulation of claim 42, wherein when R¹ and/or R³ is alkyl, the R¹ and/or R³ is methyl.

44. The formulation of claim 42, wherein when R¹ and/or R² is an aralkyl moiety, the aryl of the aralkyl moiety is phenyl and the alkyl of the aralkyl moiety is methyl.

45. The formulation of claim 42, wherein when R¹ and/or R² is a heteroaralkyl moiety, the heteroaryl of the heteroaralkyl moiety is pyridinyl.

46. The formulation of claim 42, wherein the second compound has a structure wherein, R¹ is hydrogen, C₁-C₄-alkyl, benzyl or 3-(pyridin-3-yl)propyl;
R² is hydrogen, benzyl or 2-(6-methylpyridin-3-yl)ethyl);
R³ is hydrogen, C₁-C₄-alkyl, or halogen.

47. The formulation of claim 43, wherein R¹ is C₁-C₄-alkyl, benzyl or 3-(pyridin-3-yl)propyl.

48. The formulation of claim 47, wherein when R¹ and/or R³ is C₁-C₄-alkyl, the C₁-C₄-alkyl is unsubstituted.

49. The formulation of claim 43, wherein the compound is selected from the group consisting of dimebolin (2,8-dimethyl-5-(2-(6-methylpyridin-3-yl)ethyl)-2,3,4,5-tetrahydro-1H-pyrido[4,3-b]indole); 8-chloro-2-methyl-5-(2-(6-methylpyridin-3-yl)ethyl)-2,3,4,5-tetrahydro-1H-pyrido[4,3-b]indole; mebhydroline (5-benzyl-2-methyl-2,3,4,5-tetrahydro-1H-pyrido[4,3-b]indole); 2,8-dimethyl-1,3,4,4a,5,9b-hexahydro-1H-pyrido[4,3-b]indole; 8-fluoro-2-(3-(pyridin-3-yl)propyl)-2,3,4,5-tetrahydro-1H-pyrido[4,3-b]indole; and 8-methyl-1,3,4,4a,5,9b-tetrahydro-1H-pyrido[4,3-b]indole.

50. The formulation of claim 42, wherein the compound is dimebolin (2,8-dimethyl-5-(2-(6-methylpyridin-3-yl)ethyl)-2,3,4,5-tetrahydro-1H-pyrido[4,3-b]indole).

51. The formulation of claim 42, wherein the formulation further comprises a pharmaceutically acceptable excipient.

52. The formulation of claim 42, wherein the first compound is effective against haemoglobinopathy or a disease or disorder caused by a defect in a gene encoding a mitochondrial protein or tRNA.

53. The formulation of claim 42, wherein the first compound is selected from the group consisting of: vitamin, antioxidant compound, iron chelator, antioxidant used to reduce preferryl-Hb, indicaxanthin, a drug used to lower lung hypertension, Gardos channel blocker, a drug used to modify hemoglobin switching, a drug used to treat vaso-occlusive crises, analgesic, NSAID, opiod, and antibiotic.

54. The formulation of claim 42, wherein the first compound is selected from the group consisting of erythropoietin, erythropoietin mutant, erythropoietin biosimilar, erythropoietin mimetic, Coenzyme Q, vitamin E, Idebenone, MitoQ, deferoxamine, deferasirox, indicaxanthin, sildenafil, nifedine, hydroxyurea, seniapoc, phytochemical, nicosan; folic acid, quinolone and macrolide.
